(19) United States
(12) Patent Application Publication
(10) Pub. No.: US 2002/0055456 A1
(43) Pub. Date: May 9, 2002

(54) Therapeutic methods that target fractalkine or CX3CR1

(75) Inventor: Alisa E. Koch, River Forest, IL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Northwestern University, Evanston, IL

(21) Appl. No.: 09/789,486

(22) Filed: Feb. 20, 2001

Related U.S. Application Data
(60) Provisional application No. 60/183,568, filed on Feb. 18, 2000.

(51) Int. Cl. A61K 31/00; A61K 39/395

(52) U.S. Cl. 514/1; 424/143.1

(57) ABSTRACT

The invention relates to antagonists of CX3C chemokine receptor 1 (CX3CR1) function, antagonists of fractalkine function and to therapeutic methods employing the antagonists. The invention also relates to a method for diagnosing rheumatoid arthritis.

### RELATED APPLICATIONS

This application claims the benefit of U.S. Provisional Application No. 60/183,568, filed Feb. 18, 2000, the entire teachings of which are incorporated herein by reference.

### GOVERNMENT SUPPORT

The invention was supported by grants AR30692, AR41492 and AI40987 from National Institutes of Health (U.S.A.) and by funds from the Veteran's Administration Research Service. The Government has certain rights in the invention.

### BACKGROUND OF THE INVENTION

Inflammatory arthritis includes several discrete diseases of the joint where the immune system is causing or exacerbating inflammation in the joint. Rheumatoid arthritis is a common type of inflammatory arthritis. The initial symptoms of rheumatoid arthritis and other types of inflammatory arthritis include pain and swelling of one or more joints and persistent morning stiffness. Early diagnosis of the disease is difficult and many cases are only diagnosed when the disease has progressed sufficiently to produce characteristic clinical features which frequently become apparent 1 or 2 years after the onset of disease.

Rheumatoid arthritis produces characteristic changes in the joints. Included among these is hyperplasia and hypertrophy of synovial lining cells, vascular changes including angiogenesis (e.g., neovascularization) and infiltration of the joint by leukocytes. The rheumatoid synovium contains a variety of inflammatory mediators which are produced by activated leukocytes and fibroblasts, including cytokines and chemokines produced locally.

Current therapy is directed toward relieving the symptoms of the disease and is largely empirical. None of the existing therapeutic interventions is curative and despite therapeutic intervention, many individuals are still crippled by the disease.

Thus, a need exists for a method for diagnosing rheumatoid arthritis, and for new methods for treating inflammatory arthritis and inhibiting angiogenesis.

### SUMMARY OF THE INVENTION

The invention relates to antagonists of CX3C chemokine receptor 1 (CX3CR1) function, antagonists of fractalkine (fkn) function and to therapeutic methods employing the antagonists. In one aspect, the invention is a method of treating a subject having inflammatory arthritis. In one embodiment, the method comprises administering a therapeutically effective amount of an antagonist of CX3CR1 function to a subject having inflammatory arthritis. In another embodiment, the method of treating a subject having inflammatory arthritis comprises administering to the subject a therapeutically effective amount of an antagonist of fractalkine function. In preferred embodiments, the method is a method of treating a subject having rheumatoid arthritis.

The antagonist (i.e., antagonist of CX3CR1 function, antagonist of fractalkine function) to be administered can be an agent such as a protein, peptide, peptidomimetic, natural product or small organic molecule, that inhibits (reduces, prevents) one or more functions of CX3CR1 or fractalkine.

In another aspect, the invention is a method of inhibiting angiogenesis in a subject comprising administering a therapeutically effective amount of an antagonist of CX3CR1 function and/or an antagonist of fractalkine function to a subject in need thereof. The method of inhibiting angiogenesis can be used to inhibit (reduce or prevent) pathogenic neovascularization, such as that associated with cancers (e.g., tumor formation and growth), retinopathy (e.g., retinopathy of prematurity, diabetic retinopathy), retinal vein occlusion, macular degeneration (e.g., age-related macular degeneration), hemangiomas, inflammatory arthritis (e.g., rheumatoid arthritis) and psoriasis.

In another aspect, the invention is a method of diagnosing rheumatoid arthritis. The method comprises determining the amount of soluble fractalkine contained in a sample of synovial fluid obtained from a subject suspected of having rheumatoid arthritis and comparing the determined amount to a suitable control. An elevated amount of soluble fractalkine in the synovial fluid from the subjected suspected of having rheumatoid arthritis relative to a suitable control is indicative of rheumatoid arthritis.

### BRIEF DESCRIPTION OF THE DRAWINGS

FIGS. 1A-1D are graphs illustrating the percentage of synovial tissue (ST) cells that express fractalkine (fkn) or CX3C chemokine receptor 1 (CX3CR1) and the circumference of the joint over time in adjuvant-induced arthritis (AIA) in rats. FIG. 1A shows that the greatest percentage of fibroblasts and endothelial cells that stained positively for fractalkine were detected on days 18 and 25 following administration of adjuvant. FIG. 1B shows that a significant percentage of macrophages and lymphocytes also stained positively for fractalkine on days 18 and 25. FIG. 1C shows that fibroblasts in the ST constitutively expressed CX3CR1 and that endothelial cells did not stain positively for the receptor. FIG. 1D shows that macrophages in the ST constitutively expressed CX3CR1 and that lymphocytes did not stain positively for the receptor. The data presented in FIGS. 1A-1D are presented as mean+S.E., n=3, Circ=ankle circumference.

FIGS. 2A and 2B are histograms illustrating the expression of fractalkine (fkn) or CX3CR1 on dendritic cells in synovial tissues from rat AIA 18 and 25 days following administration of adjuvant. FIG. 2A shows the percentage of dendritic cells that stained positively for fractalkine. FIG. 2B shows the percentage of dendritic cells that stained positively for CX3CR1. The percentage of dendritic cells in synovial tissue isolated from healthy rats that expressed fkn or CX3CR1 is also shown in FIGS. 2A and 2B (day 0). The percentage of dendritic cells that stained positively for fkn or CX3CR1 increased during the period of maximal inflammation in the rat joint (days 18-25).

FIG. 3A is a histogram illustrating the percentage of monocytes (CD14⁺) isolated from peripheral blood (PB) or synovial fluid (SF) that expressed fractalkine (fkn) or CX3CR1. The peripheral blood and synovial fluid were obtained from humans with rheumatoid arthritis and expression of fkn, CX3CR1 and CD14 was assessed by flow cytometry. The percentage of monocytes expressing fkn was similar in PB and SF. There was no statistical difference in the percentage of monocytes expressing CX3CR1 in the PB or SF. The percentage of monocytes expressing either CX3CR1 or fkn exceeded that of T-cells in PB and SF. Data are presented as the mean±S.E.

FIG. 3B is a histogram illustrating the percentage of T cells (CD3⁺) isolated from peripheral blood (PB) or synovial fluid (SF) that expressed fractalkine (fkn) or CX3CR1. The peripheral blood and synovial fluid were obtained from humans with rheumatoid arthritis and expression of fkn, CX3CR1 and CD3 was assessed by flow cytometry. The percentages of CD3⁺ T-cells expressing fkn in PB and SF were similar. However, the percentage of CD3⁺ T-cells expressing CX3CR1 was significantly higher in RA PB than SF. Data are presented as the mean±S.E.

FIG. 4 is a histogram illustrating the amount of soluble fractalkine (sfkn) contained in samples of rheumatoid arthritis synovial fluid (RA SF), osteoarthritis synovial fluid (OA SF), pooled synovial fluids obtained from individuals having other types of arthritis (juvenile rheumatoid arthritis, psoriatic arthritis, polyarthritis, spondyloarthropathy, inflammatory myopathy and gout) (Other SF), serum from healthy donors (NL Sera) and serum from individuals with arthritic disease (rheumatoid arthritis, osteoarthritis, juvenile rheumatoid arthritis, psoriatic arthritis, polyarthritis, and gout) (Disease Sera). Soluble fkn was measured by ELISA. RA SF samples contained significantly elevated levels of sfkn compared to all other SFs and sera measured. SF from OA and other diseases showed similar sfkn levels as normal sera. Similarly, sera from patients diagnosed with arthritic diseases contained a mean of 0.71 ng/ml sfkn. Results are presented as mean+S.E.

FIG. 5 is a histogram demonstrating that immunodepletion of soluble fractalkine from synovial fluid from patients with rheumatoid arthritis inhibited the chemotactic response of monocytes upon stimulation with the synovial fluid. Synovial fluid samples were obtained from four individuals (1, 2, 3, 4) with rheumatoid arthritis. The samples were immunodepleted of soluble fkn using a goat anti-human fkn IgG (anti-fkn) or were immunodepleted using a nonspecific goat IgG (IgG). The number of monocytes that migrated toward the synovial fluid samples in an in vitro chemotaxis assay are shown. All synovial fluid samples that were depleted of soluble fkn induced significantly less chemotaxis compared to the synovial fluid samples that were depleted using the nonspecific IgG antibody. Hanks balanced saline solution (HBSS) was the negative control and N-formyl-methionyl-leucyl-phenylalanine (fMLF) was the positive control. The presented data are means±S.E.

FIGS. 6A and 6B illustrate a nucleotide sequence encoding human (Homo sapiens) fractalkine (SEQ ID NO:1) deposited in Genbank under Accession Number NM_{—}002996, having an open-reading frame beginning at position 80. Nucleotides 80-151 encode the signal peptide, nucleotides 152-1270 encode the mature peptide, nucleotides 152-379 encode the chemokine module, nucleotides 380-1102 encode the glycosylation stalk, nucleotides 1103-1159 encode the transmembrane helix and nucleotides 1160-1270 encode the intracellular domain. The teachings of the Genbank deposit under Accession Number NM_{—}002996 are incorporated herein by reference in their entirety.

FIG. 7 illustrates the amino acid sequence of a human fractalkine protein (SEQ ID NO:2) encoded by the DNA sequence shown in FIGS. 6A and 6B (SEQ ID NO:1).

FIG. 8 illustrates a nucleotide sequence encoding human (Homo sapiens) CX3C chemokine receptor 1 (CX3CR1, SEQ ID NO:3) deposited in Genbank under Accession Number NM 001337, having an open-reading frame beginning at position 46. The teachings of the Genbank deposit under Accession Number NM_{—}001337 are incorporated herein by reference in their entirety.

FIG. 9 illustrates the amino acid sequence of a human CX3CR1 protein (SEQ ID NO:4) encoded by the DNA sequence shown in FIG. 8 (SEQ ID NO:3).

FIG. 10 is a histogram showing that the expression of fractalkine (fkn) and CX3CR1 in ankle homogenates from rats in which adjuvant-induced arthritis (AIA) had been induced, was significantly upregulated 18 days after administration of adjuvant (*p<0.05, significantly different from expression at day 0). Expression of rat fkn or CX3CR1 was determined by RT-PCR of total RNA obtained from rat ankle homogenates using primers specific for fkn or CX3CR1. The PCR products were separated by agarose gel electrophoresis and visualized by staining. The gels were scanned and anayzed by densitometry. The histogram shows densitometric analysis of the expression of fkn and CX3CR1 normalized to the housekeeping control gene, glyceraldehyde-3-phosphate dehydrogenase (GAPDH).

FIG. 11 is a graph demonstrating that fractalkine (fkn) induced human dermal microvascular endothelial cell (HMVEC) chemotaxis in a concentration-dependent manner in an in vitro assay. Results of a representative experiment of four are depicted and the data points represent the mean number of cells that migrated (HMVECs/well±SE). Fkn, in the 10⁻⁶ to 10² nM range, significantly increased HMVEC chemotaxis over negative control phosphate-buffered saline (PBS) (*p<0.05, significantly different from PBS control). Also shown is HMVEC chemotaxis for the known potent endothelial cell chemoattractant, basic fibroblast growth factor (bFGF, 60 nM).

FIG. 12A is a histogram demonstrating that a goat anti-human fractalkine IgG antibody (anti-fkn) inhibited fkn-induced human dermal microvascular endothelial cell (HMVEC) chemotaxis in an in vitro assay. Results of a representative experiment of three are depicted and the data points represent the mean number of cells that migrated (HMVECs/well+SE). At concentrations of 1 pM or 10 nM of fkn, addition of anti-fkn antibody significantly inhibited fkn-induced HMVEC chemotaxis (*p<0.05, significantly different from goat IgG control antibody). Basic fibroblast growth factor (bFGF) (60 nM) and phosphate-buffered saline (PBS) were used as positive and negative controls respectively, for the assay.

FIG. 12B is a histogram demonstrating that a goat anti-human fractalkine IgG antibody (anti-fkn) did not inhibit basic fibroblast growth factor (bFGF)-induced human dermal microvascular endothelial cell (HMVEC) chemotaxis in an in vitro assay. Results of a representative experiment of three are depicted and the data points represent the mean number of cells that migrated (HMVECs/well±SE). At a concentration of 60 nM of bFGF, addition of anti-fkn antibody did not inhibit bFGF-induced HMVEC chemotaxis as compared to a goat IgG control antibody (goat IgG). Phosphate-buffered saline (PBS) was used as a negative control for the assay.

FIG. 13 is a graph demonstrating that fractalkine (fkn), at concentrations of 10⁻¹⁰ to 10² nM, did not induce human dermal microvascular endothelial cell (HMVEC) proliferation. Results of a representative experiment of four are depicted and the data points represent the number of viable cells/well (measured by absorbance at a wavelength of 490 nm of quadruplicate wells±SE). Basic fibroblast growth factor (bFGF) (60 nM) and endothelial basal media (EBM)+2% fetal bovine serum (FBS) were used as positive and negative controls respectively, for the assay.

FIG. 14A is a histogram illustrating that fractalkine (fkn) induced human dermal microvascular endothelial cell (HMVEC) tube formation on Matrigel™ in vitro. Values represent the mean number of HMVEC tube branches/well±SE for 3 or 4 assays, as counted by a blinded observer. Fkn and phorbol myristate acetate (PMA, 50 nM) both significantly induced HMVEC tube formation relative to their negative controls (PBS=phosphate-buffered saline, DMSO=dimethyl sulfoxide+PBS)(*p<0.05, significantly different from vehicle control).

FIG. 14B is a histogram illustrating that a goat anti-human fractalkine IgG antibody (anti-fkn) inhibited fkn-induced, but not phorbol myristate acetate (PMA)-induced, human dermal microvascular endothelial cell (HMVEC) tube formation on Matrigel™ in vitro. Values represent the mean number of HMVEC tube branches/well±SE for 3 or 4 assays, as counted by a blinded observer. At a concentration of 10 nM of fkn, addition of anti-fkn antibody (25 μg/ml) significantly inhibited HMVEC tube formation as compared to a goat IgG control antibody (IgG, 25 μg/ml )(*p<0.05, significantly different from IgG control antibody). Addition of anti-fkn antibody did not inhibit HMVEC tube formation induced by 50 nM of PMA.

FIG. 15A is a histogram illustrating that fractalkine (fkn) induced angiogenesis in Matrigel™ plugs in vivo. Female mice were injected subcutaneously with 0.5 ml of Matrigel™ combined with either phosphate-buffered saline (PBS) or fkn (100 nM). 7-10 days later, the plugs were removed and the amount of hemoglobin in the plug, which correlates with the number of blood vessels in the plug, was determined. Values represent the concentration of hemoglobin (g/dl)/Matrigel™ plug weight (mg)±SE for 18 assays. Fkn induced significantly more blood vessels in the Matrigel™ plugs than did negative control phosphate-buffered saline (PBS) (0.77±0.15 versus 0.33±0.08 g/dl of hemoglobin/mg of plug weight respectively, n=18, *p<0.05, significantly different from vehicle control).

FIG. 15B is a histogram illustrating that fractalkine (fkn) is as potent an inducer of angiogenesis in vivo as interleukin-8 (IL-8) and epithelial neutrophil-activating protein (ENA-78). Female mice were injected subcutaneously with 0.5 ml of Matrigel™ combined with either fkn (100 nM), IL-8 (100 nM) or ENA-78 (100 nM). 7-10 days later, the plugs were removed and hemoglobin content was determined. Values represent the concentration of hemoglobin ((g/dl)/Matrigel™ plug weight (mg)) as a percentage of the concentration of hemoglobin ((g/dl)/Matrigel™ plug weight (mg)) induced by the known angiogenic factor, bovine acidic fibroblast growth factor (aFGF). Fkn exhibited 78% of the angiogenic potency of aFGF, while IL-8 and ENA-78 exhibited 65% and 44% respectively (n=7-9).

FIG. 16 is a histogram demonstrating that the in vivo angiogenic activity of synovial fluid (SF) from patients with rheumatoid arthritis (RA) was decreased by immunodepletion of fractalkine (fkn). SFs from six RA patients were pooled and either incubated with 25 μg/ml of goat anti-human fractalkine IgG antibody (SF+anti-fkn) or goat IgG control antibody (SF+IgG). These immunodepleted SFs were then diluted in Matrigel™ and injected subcutaneously into mice. 7-10 days later, the plugs were removed and hemoglobin content was determined. Values represent the mean concentration of hemoglobin (g/dl)/Matrigel™ plug weight (mg)±SE. Angiogenesis induced by the pooled RA SFs was significantly decreased by immunodepleting fkn as compared to RA SFs which were immunodepleted using an IgG control antibody (0.028±0.02 versus 1.38±0.57 g/dl of hemoglobin/mg of plug weight respectively, n=12, *p<0.05, significantly different from IgG control antibody).

FIG. 17 is a histogram showing that the in vivo angiogenic activity of synovial tissue (ST) homogenates from patients with rheumatoid arthritis (RA) was decreased by immunodepletion of fractalkine (fkn). ST homogenates from five RA patients were pooled and either incubated with 25 μg/ml of goat anti-human fractalkine IgG antibody (ST+anti-fkn) or goat IgG control antibody (ST+IgG). These immunodepleted ST homogenates were then diluted in Matrigel™ and injected subcutaneously into mice. 7-10 days later, the plugs were removed and hemoglobin content was determined. Values represent the mean concentration of hemoglobin (g/dl)/Matrigel™ plug weight (mg)±SE. Angiogenesis induced by the pooled RA ST homogenates was significantly decreased by immunodepleting fkn as compared to RA ST homogenates which were immunodepleted using an IgG control antibody (0.09±0.08 versus 0.66±0.12 g/dl of hemoglobin/mg of plug weight respectively, n=12, *p<0.05, significantly different from IgG control antibody).

### DETAILED DESCRIPTION OF THE INVENTION

Chemokines are a family of proinflammatory mediators that promote recruitment and activation of multiple lineages of leukocytes (e.g., lymphocytes, macrophages). They can be released by many kinds of tissue cells after activation. Continuous release of chemokines at sites of inflammation can mediate the ongoing migration and recruitment of effector cells to sites of chronic inflammation. The chemokines are related in primary structure and share four conserved cysteines, which form disulfide bonds. Based upon this conserved cysteine motif, the family can be divided into distinct branches, including the C-C chemokines (β-chemokines), C-X-C chemokines (α-chemokines), and the C-XXX-C chemokines (CX3C chemokines), in which the first two conserved cysteines are adjacent or separated by one or three intervening residues, respectively (see e.g., Baggiolini, M. and Dahinden, C. A., Immunology Today, 15:127-133 (1994); Bazan, J. F. et al., Nature, 385:640-644 (1997)).

The C-C chemokines include, for example, RANTES (Regulated on Activation, Normal T Expressed and Secreted), the macrophage inflammatory proteins 1α and 1β (MIP-1α and MIP-1β), cotaxin and human monocyte chemotactic proteins 1-3 (MCP-1, MCP-2, MCP-3), which have been characterized as chemoattractants and activators of monocytes or lymphocytes. The C-X-C chemokines include a number of potent chemoattractants and activators of neutrophils, such as interleukin 8 (IL-8), platelet factor four (PF4) and neutrophil-activating peptide-2 (NAP-2). The CX3C chemokines include fractalkine, which is also referred to as neurotactin (Pan, Y. et al., Nature, 387:611-617 (1997)), CX3CL1, CXXXCL1, ABCD-3 (Schaniel C., et al., Eur. J. Immunol., 29:2934-2947 (1999)) and SCYD1 (Nomiyama H. et al, Cytogenet. Cell Genet., 81:10-11 (1998)).

Chemokines, such as RANTES and MIP-1α, for example, have been implicated in human acute and chronic inflammatory diseases including respiratory diseases, such as asthma and allergic disorders and inflammatory arthritis (e.g., rheumatoid arthritis). For example, the CXC chemokines interleukin-8 (IL-8) (Endo, H. et al., Lymphokine Cytokine Res., 10:245 (1991); Koch, A. et al., J. Immunol., 147:2187 (1991); Rampart, M. et al., Lab. Invest., 66:512 (1992); Deleuran, B. et al., Scand. J. Rheumatol., 23:2 (1994); Peichl, P. et al., Ann. Rheum. Dis. 51:19 (1992); Peichl, P. et al., Scand. J. Immunol., 34:333 (1991); Seitz, M. et al., J. Clin. Invest., 87:463 (1991); Brennan, F. et al., Eur. J. Immunol., 20:2141 (1990); Symons, J. et al., Scand. J. Rheumatol, 21:92 (1992)) and epithelial neutrophil activating peptide-78 (ENA-78) (Koch, A. et al., J. Clin. Invest., 94:1012 (1994)), and the CC chemokines MCP-1(Koch, A. et al., J. Clin. Invest., 90:772 (1992); Akahoshi, T. et al., Arthritis Rheum., 36:762 (1993)), MIP-1α (Koch, A. et al., J. Clin. Invest., 93:921 (1994)) and RANTES (Schall, T. et al., Nature, 347:669 (1990); Rathanaswami, P. et al., J. Biol. Chem., 268:5834 (1993); Hosaka, S. et al., Clin. Exp. Immunol., 97:451 (1994); Volin, M. et al., Clin. Immunol. Immunopathol., 89:44 (1998)) are reported to be mediators of inflammation in rheumatoid arthritis. In addition to recruiting cells to sites of active inflammation, certain chemokines (e.g., IL-8) can promote or induce angiogenesis (Koch, A. et al., Science, 258:1798 (1992); Strieter, R. et al., Am. J. Pathol., 141:1279 (1992); Hu, D. et al., Inflammation, 17:135 (1993)).

The CX3C chemokine fractalkine (fkn) is a transmembrane molecule that has an extra-cellular region containing a conserved chemokine domain atop a mucin-like stalk (Imai, T. et al., Cell, 91:521 (1997)). A soluble form of fractalkine, which is believed to be produced by processing (e.g., proteolytic cleavage) of the transmembrane molecule, is produced by cells in vivo and in vitro. Fractalkine can function as a cellular adhesion molecule and as a chemoattractant for monocytes and lymphocytes (Imai, T. et al., Cell, 91:521 (1997); Kanazawa, N. et al., Eur. J. Immunol., 29:1925 (1999); Fong, A. et al., J. Exp. Med., 188:1413 (1998); Bazan, J. et al., Nature, 385:640 (1997)).

The chemokine receptors are members of a superfamily of G protein-coupled receptors (GPCRs) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). Conserved features include seven hydrophobic domains spanning the plasma membrane, which are connected by hydrophilic extracellular and intracellular loops. The majority of the primary sequence homology occurs in the hydrophobic transmembrane regions with the hydrophilic regions being more diverse.

The human CX3C chemokine receptor 1 (CX3CR1, also referred to as CXXXCR1 and V28 (Raport, C. J. et al., Gene, 163:295-299 (1995); WO 94/12635; Godiska et al., U.S. Pat. No. 5,759,804, the entire teachings of which are incorporated herein by reference) can bind fractalkine and is expressed by a variety of different cells and tissues including peripheral blood leukocytes (PBL), spleen and brain (Raport, C. J., et al., Gene, 163:295-299 (1995)).

To determine if CX3CR1 and/or fractalkine function is involved in the initiation, progression and/or maintenance of inflammatory arthritis, a study analyzing the expression of the chemokine and the receptor in adjuvant induced arthritis in rats (Rattus norvegicus), an accepted model of rheumatoid arthritis in human beings (Homo sapiens), was conducted. Additional studies analyzed the expression and activity of fractalkine and CX3CR1 in synovial fluid, synovial tissue and plasma obtained from humans having rheumatoid arthritis (Example 1). As described herein, fractalkine and CX3CR1 expression was detected in synovial tissue removed from both rats and humans by immunohistochemical staining of tissue sections. In studies of rat adjuvant induced arthritis (AIA), a high percentage of synovial tissue macrophages and fibroblasts expressed fractalkine (fkn) and CX3CR1. A large percentage of synovial tissue endothelial cells stained positively for fkn, and synovial tissue dendritic cells stained positively for fkn and CX3CR1. The percentage of macrophages and fibroblasts that stained positively for CX3CR1 increased throughout the study period (through day 54). The expression kinetics of CX3CR1 and fkn in rat AIA were similar. The percentage of cells which stained positively for fkn and/or CX3CR1 was noticeably elevated in tissues removed from rats 18 and 25 day after administration of adjuvant. In addition, an increased percentage of synovial tissue dendritic cells stained positively for fkn and CX3CR1 on days 18 and 25 in rat AIA. Maximal inflammation and cellular recruitment into the joint in this model occurs during the period of from about 18 days to about 25 days after administration of adjuvant. Thus, these data clearly illustrate the relationship between the appearance of cells that express CX3CR1 and/or fractalkine and the course (i.e., onset and severity) of disease in rat AIA.

Immunohistochemical studies of synovial tissue removed from humans with rheumatoid arthritis yielded similar results. The human synovial tissues stained positively for CX3CR1 and the majority of cells in the synovial lining layer were intensely positive for CX3CR1 expression. High expression of CX3CR1 was detected on tissue macrophages and endothelium. The synovial tissue endothelium and synovial lining stained positively for fkn.

The expression of fractalkine and CX3CR1 on peripheral blood cells and synovial fluid cells isolated from humans with rheumatoid arthritis was analyzed by flow cytometry. This analysis revealed that fractalkine and CX3CR1 were expressed to varying degrees on monocytes (CD14⁺) and T cells (CD3⁺) isolated from both peripheral blood and synovial fluid. A greater percentage of peripheral blood T cells expressed CX3CR1 than did synovial fluid T cells, and a high percentage of monocytes isolated from both synovial fluid and peripheral blood expressed both flkn and CX3CR1.

In further studies, the amount of the soluble form of fractalkine (sfkn) contained in synovial fluid and serum samples obtained from humans with rheumatoid arthritis or other arthritic diseases (e.g., osteoarthritis, juvenile rheumatoid arthritis, psoriatic arthritis, polyarthritis, spondyloarthropathy, inflammatory myopathy, gout) and in serum samples obtained from healthy donors was quantified. Synovial fluids obtained from humans with rheumatoid arthritis contained significantly elevated quantities of sfkn compared to all other synovial fluids assessed. The quantity of sfkn in synovial fluids from rheumatoid arthritis was also significantly elevated in comparison to the quantity contained in sera of healthy or arthritic donors. The sfkn contained in the rheumatoid synovial fluid was biologically active and induced chemotaxis of monocytes in an in vitro assay. Furthermore, the sfkn-induced chemotaxis of monocytes was inhibited by immunodepletion of sflm from the synovial fluid using an anti-fractalkine antibody.

The studies described herein clearly demonstrate a correlation between the appearance of cells that express CX3CR1 and/or fractalkine in synovial tissue and the course of disease in an established animal model of rheumatoid arthritis. The studies also demonstrate that the amount of soluble fractalkine contained in the synovial fluid of arthritic joints can be used to distinguish rheumatoid arthritis from other arthritic diseases. Furthermore, the studies demonstrate that soluble fractalkine in the synovial fluid of rheumatoid joints is biologically active and can induce chemotaxis of cells expressing CX3CR1.

Also described herein are the results of a study (Example 2) which demonstrate that fractalkine can induce angiogenesis. The results of the study implicate fractalkine and its receptors (e.g., CX3CR1) in the pathogenic vasculoproliferation found a variety of conditions (e.g., rheumatoid arthritis, cancer).

Agents which inhibit the activity of CX3CR1 and/or fractalkine can inhibit cellular responses (e.g., activation, migration, adhesion) mediated by the receptor and/or chemokine and can inhibit the initiation, progression and/or maintenance of inflammatory arthritis (e.g., rheumatoid arthritis) and angiogenesis. Accordingly, the invention relates to therapeutic methods for treating a subject having inflammatory arthritis. The invention also relates to a method for inhibiting angiogenesis in a subject in need thereof, such as a subject having inflammatory arthritis (e.g., rheumatoid arthritis) or a tumor (e.g., solid tumor). The methods of the invention comprise administering an effective amount of an (i.e., one or more) antagonist of CX3CR1 function and/or an antagonist of fractalkine function to a subject in need thereof.

CX3CR1 Antagonists

As used herein, the term “antagonist of CX3CR1 function” refers to an agent (e.g., a molecule, a compound) which can inhibit a (i.e., one or more) function of CX3CR1. For example, an antagonist of CX3CR1 function can inhibit the binding of one or more ligands (e.g., fractalkine) to CX3CR1 and/or inhibit signal transduction mediated through CX3CR1 (e.g., GDP/GTP exchange by CX3CR1-associated G proteins, intracellular calcium flux). Accordingly, CX3CR1-mediated processes and cellular responses (e.g., proliferation, migration, chemotactic responses, secretion or degranulation) can be inhibited with an antagonist of CX3CR1 function.

Preferably, the antagonist of CX3CR1 function is a compound which is, for example, a small organic molecule, natural product, protein (e.g., antibody, chemokine, cytokine), peptide or peptidomimetic. Several types of molecules that can be used to antagonize one or more functions of chemokine receptors are known in the art, including small organic molecules, proteins, such as antibodies (e.g., polyclonal sera, monoclonal, chimeric, humanized) and antigen-binding fragments thereof (e.g., Fab, Fab′, F(ab′)₂, Fv); chemokine mutants and analogues, for example, vMIP-II (Chen, S. et al., J. Exp. Med., 188:193-198 (1998)) those disclosed in U.S. Pat. No. 5,739,103 issued to Rollins et al., WO 96/38559 by Dana Farber Cancer Institute and WO 98/06751 by Research Corporation Technologies, Inc.; and peptides, for example, those disclosed in WO 98/09642 by The United States of America. The entire teachings of each of the above cited patent applications and references is incorporated herein by reference.

Antagonists of CX3CR1 function can be identified, for example, by screening libraries or collections of molecules, such as the Chemical Repository of the National Cancer Institute, as described herein or using other suitable methods. Antagonists thus identified can be used in the therapeutic methods described herein.

Another source of antagonists of CX3CR1 function are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

The term “natural product”, as used herein, refers to a compound which can be found in nature, for example, naturally occurring metabolites of marine organisms (e.g., tunicates, algae) or other organisms or plants and which possess biological activity, e.g., can antagonize CX3CR1 function. For example, lactacystin, paxlitaxol and cyclosporin A are natural products which can be used as anti-proliferative or immunosuppressive agents.

Natural products can be isolated and identified by suitable means. For example, a suitable biological source (e.g., vegetation) can be homogenized (e.g., by grinding) in a suitable buffer and clarified by centrifugation, thereby producing an extract. The resulting extract can be assayed for the capacity to antagonize CX3CR1 function, for example, by the assays described herein. Extracts which contain an activity that antagonizes CX3CR1 function can be further processed to isolate the CX3CR1 antagonist by suitable methods, such as, fractionation (e.g., column chromatography (e.g., ion exchange, reverse phase, affinity), phase partitioning, fractional crystallization) and assaying for biological activity (e.g., antagonism of CX3CR1 activity). Once isolated, the structure of a natural product can be determined (e.g., by nuclear magnetic resonance (NMR)) and those of skill in the art can devise a synthetic scheme for synthesizing the natural product. Thus, a natural product can be isolated (e.g., substantially purified) from nature or can be fully or partially synthetic. A natural product can be modified (e.g., derivatized) to optimize its therapeutic potential. Thus, the term “natural product”, as used herein, includes those compounds which are produced using standard medicinal chemistry techniques to optimize the therapeutic potential of a compound which can be isolated from nature.

The term “peptide”, as used herein, refers to a compound consisting of from about two to about ninety amino acid residues wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are know in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can antagonize CX3CR1 function. Such peptide antagonists can then be isolated by suitable methods.

The term “peptidomimetic”, as used herein, refers to molecules which are not polypeptides, but which mimic aspects of their structures. For example, polysaccharides can be prepared that have the same functional groups as peptides which can antagonize CX3CR1. Peptidomimetics can be designed, for example, by establishing the three dimensional structure of a peptide agent in the environment in which it is bound or will bind to CX3CR1. The peptidomimetic comprises at least two components, the binding moiety or moieties and the backbone or supporting structure.

The binding moieties are the chemical atoms or groups which will react or form a complex (e.g., through hydrophobic or ionic interactions) with CX3CR1, for example, with the amino acid(s) at or near the ligand binding site. For example, the binding moieties in a peptidomimetic can be the same as those in a peptide antagonist of CX3CR1. The binding moieties can be an atom or chemical group which reacts with the receptor in the same or similar manner as the binding moiety in a peptide antagonist of CX3CR1. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more —CONH— groups for a —NHCO— group). In yet another embodiment, the peptide backbone can be substituted with a polysilane backbone.

These compounds can be manufactured by known methods. For example, a polyester peptidomimetic can be prepared by substituting a hydroxyl group for the corresponding α-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the chemical structure of the peptidomimetic.

Peptidomimetics can be synthesized and assembled into libraries comprising a few to many discrete molecular species. Such libraries can be prepared using well-known methods of combinatorial chemistry, and can be screened as described herein to determine if the library comprises one or more peptidomimetics which antagonize CX3CR1 function. Such peptidomimetic antagonists can then be isolated by suitable methods.

In one embodiment, the CX3CR1 antagonist is an antibody or antigen-binding fragment thereof having specificity for CX3CR1. The antibody can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein also encompasses functional fragments of antibodies, including fragments of chimeric, humanized, primatized, veneered or single chain antibodies. Functional fragments include antigen-binding fragments which bind to CX3CR1. For example, antibody fragments capable of binding to CX3CR1 or portions thereof, including, but not limited to Fv, Fab, Fab′ and F(ab′)₂ fragments are encompassed by the invention. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain. Single chain antibodies, and chimeric, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for mammalian (e.g., human) CX3CR1 can be raised against an appropriate immunogen, such as isolated and/or recombinant human CX3CR1 or portions thereof (including synthetic molecules, such as synthetic peptides). Antibodies can also be raised by immunizing a suitable host (e.g., mouse) with cells that express CX3CR1, such as activated monocytes or T cells (see e.g., U.S. Pat. No. 5,440,020, the entire teachings of which are incorporated herein by reference). In addition, cells expressing recombinant CX3CR1 such as transfected cells, can be used as immunogens or in a screen for antibody which binds receptor (See e.g., Chuntharapai et al., J. Immunol., 152: 1783-1789 (1994); Chuntharapai et al., U.S. Pat. No. 5,440,021).

Preparation of immunizing antigen, and polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). When monoclonal antibodies are desired, a hybridoma is generally produced by fusing a suitable immortal cell line (e.g., a myeloma cell line such as SP2/0 or P3X63Ag8.653) with antibody producing cells. The antibody producing cells, preferably those obtained from the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library (e.g., a phage display library), or which rely upon immunization of transgenic animals (e.g., mice) capable of producing a repertoire of human antibodies (see e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807; Lonberg et al., WO97/13852).

In one embodiment, the antibody or antigen-binding fragment thereof has specificity for a mammalian CX3C chemokine receptor-1 (CX3CR1), such as human CX3CR1. In a preferred embodiment, the antibody or antigen-binding fragment can inhibit binding of a ligand (i.e., one or more ligands) to CX3CR1 and/or one or more functions mediated by CX3CR1 in response to ligand binding.

Assessment of Activity of Antagonists

The capacity of an agent (e.g., proteins, peptides, natural products, small organic molecules, peptidomimetics) to antagonize CX3CR1 function can be determined using a suitable screen (e.g., high through-put assay). For example, an agent can be tested in an extracellular acidification assay, calcium flux assay, ligand binding assay or chemotaxis assay (see, for example, Hesselgesser et al., J. Biol. Chem. 273(25):15687-15692 (1998) and WO 98/02151).

In a particular assay, membranes can be prepared from cells which express CX3CR1, such as THP-1 cells (Raport, C. J. et al., Gene, 163:295-299 (1995)(American Type Culture Collection, Manassas, Va.; Accession No. TIB202). Cells can be harvested by centrifugation, washed twice with PBS (phosphate-buffered saline), and the resulting cell pellets frozen at −70 to −85° C. The frozen pellet can be thawed in ice-cold lysis buffer consisting of 5 mM HEPES (N-2-hydroxyethylpiperazine-N′-2-ethane-sulfonic acid) pH 7.5, 2 mM EDTA (ethylenediaminetetraacetic acid), 5 μg/ml each aprotinin, leupeptin, and chymostatin (protease inhibitors), and 100 μg/ml PMSF (phenyl methane sulfonyl fluoride—also a protease inhibitor), at a concentration of 1 to 5×10⁷ cells/ml, to achieve cell lysis. The resulting suspension can be mixed well to resuspend all of the frozen cell pellet. Nuclei and cell debris can be removed by centrifugation of 400×g for 10 minutes at 4° C. The resulting supernatant can be transferred to a fresh tube and the membrane fragments can be collected by centrifugation at 25,000×g for 30 minutes at 4° C. The resulting supernatant can be aspirated and the pellet can be resuspended in freezing buffer consisting of 10 mM HEPES pH 7.5, 300 mM sucrose, 1 μg/ml each aprotinin, leupeptin, and chymostatin, and 10 μg/ml PMSF (approximately 0.1 ml per each 10⁸ cells). All clumps can be resolved using a minihomogenizer, and the total protein concentration can be determined by suitable methods (e.g., Bradford assay, Lowery assay). The membrane solution can be divided into aliquots and frozen at −70 to −85° C. until needed.

The membrane preparation described above can be used in a suitable binding assay. For example, membrane protein (2 to 20 μg total membrane protein) can be incubated with 0.1 to 0.2 nM ¹²⁵I-fractalkine with or without unlabeled competitor (e.g., fractalkine, vMIP-II (Chen, S. et al., J. Exp. Med., 188:193-198 (1998)) or various concentrations of compounds to be tested. ¹²⁵I-fractalkine can be prepared by suitable methods. The binding reactions can be performed in 60 to 100 μl of a binding buffer consisting of 10 mM HEPES pH 7.2, 1 mM CaCl₂, 5 mM MgCl₂, and 0.5% BSA (bovine serum albumin), for 60 min at room temperature. The binding reactions can be terminated by harvesting the membranes by rapid filtration through glass fiber filters (e.g., GF/B or GF/C, Packard) which can be presoaked in 0.3% polyethyleneimine. The filters can be rinsed with approximately 600 μl of binding buffer containing 0.5 M NaCl, dried, and the amount of bound radioactivity can be determined by scintillation counting.

The CX3CR1 antagonist activity of test agents (e.g., compounds) can be reported as the inhibitor concentration required for 50% inhibition (IC₅₀ values) of specific binding in receptor binding assays (e.g., using 125I-fractalkine as ligand and THP-1 cell membranes). Specific binding is preferably defined as the total binding (e.g., total cpm on filters) minus the non-specific binding. Non-specific binding is defined as the amount of cpm still detected in the presence of excess unlabeled competitor (e.g., fractalkine). If desired, membranes prepared from cells which express recombinant CX3CR1 can be used in the described assay.

The capacity of an agent to antagonize CX3CR1 function can also be determined in a leukocyte chemotaxis assay using suitable cells. Suitable cells include, for example, cell lines, recombinant cells or isolated cells which express CX3CR1 and undergo CX3CR1 ligand-induced (e.g., fractalkine-induced) chemotaxis. In one example, CX3CR1-expressing recombinant L1.2 cells (see Campbell, et al. J Cell Biol, 134:255-266 (1996)), peripheral blood mononuclear cells or THP-1 cells, can be used in a modification of a transendothelial migration assay (Carr, M. W., et al. T. A., Proc. Natl Acad Sci, USA, (91):3652 (1994)). Peripheral blood mononuclear cells can be isolated from whole blood by suitable methods, for example, density gradient centrifugation and positive or preferably negative selection with specific antibodies. The endothelial cells used in this assay are preferably the endothelial cell line, ECV 304, obtained from the European Collection of Animal Cell Cultures (Porton Down, Salisbury, U.K.). Endothelial cells can be cultured on 6.5 mm diameter Transwell culture inserts (Costar Corp., Cambridge, Mass.) with 3.0 μm pore size. Culture media for the ECV 304 cells can consist of M199+10% FCS, L-glutamine, and antibiotics. The assay media can consist of equal parts RPMI 1640 and M199 with 0.5% BSA. Two hours before the assay, 2×10⁵ ECV 304 cells can be plated onto each insert of the 24 well Transwell chemotaxis plate and incubated at 37° C. Chemotactic factors such as fractalkine (diluted in assay medium) can be added to the 24-well tissue culture plates in a final volume of 600 μL. Fractalkine is commercially available from, for example, Research Diagnostics Inc., Flanders, N.J. Endothelial-coated Transwells can be inserted into each well and 10⁶ cells of the leukocyte type being studied can be added to the top chamber in a final volume of 100 μL of assay medium. The plate can then be incubated at 37° C. in 5% CO₂/95% air for 1-2 hours. The cells that migrate to the bottom chamber during incubation can be counted, for example using flow cytometry. To count cells by flow cytometry, 500 μL of the cell suspension from the lower chamber can be placed in a tube and relative counts can obtained for a set period of time, for example, 30 seconds. This counting method is highly reproducible and allows gating on the leukocytes and the exclusion of debris or other cell types from the analysis. Alternatively, cells can be counted with a microscope. Assays to evaluate chemotaxis inhibitors can be performed in the same way as control experiment described above, except that antagonist solutions, in assay media containing up to 1% of DMSO co-solvent, can be added to both the top and bottom chambers prior to addition of the cells. Antagonist potency can be determined by comparing the number of cell that migrate to the bottom chamber in wells which contain antagonist, to the number of cells which migrate to the bottom chamber in control wells. Control wells can contain equivalent amounts of DMSO, but no antagonist.

The activity of an antagonist of CX3CR1 function can also be assessed by monitoring cellular responses induced by active receptor, using suitable cells expressing receptor. For instance, exocytosis (e.g., degranulation of cells leading to release of one or more enzymes or other granule components, such as esterases (e.g., serine esterases), perforin, and/or granzymes), inflammatory mediator release (such as release of bioactive lipids such as leukotrienes (e.g., leukotriene C₄)), and respiratory burst, can be monitored by methods known in the art or other suitable methods (see e.g., Taub, D. D. et al., J. Immunol., 155: 3877-3888 (1995), regarding assays for release of granule-derived serine esterases; Loetscher et al., J. Immunol., 156: 322-327 (1996), regarding assays for enzyme and granzyme release; Rot, A. et al., J. Exp. Med., 176: 1489-1495 (1 992) regarding respiratory burst; Bischoff, S. C. et al., Eur. J. Immunol., 23: 761-767 (1993) and Baggliolini, M. and C. A. Dahinden, Immunology Today, 15: 127-133 (1994)).

In one embodiment, an antagonist of CX3CR1 is identified by monitoring the release of an enzyme upon degranulation or exocytosis by a cell capable of this function. Cells expressing CX3CR1 can be maintained in a suitable medium under suitable conditions, and degranulation can be induced. The cells are contacted with an agent to be tested, and enzyme release can be assessed. The release of an enzyme into the medium can be detected or measured using a suitable assay, such as in an immunological assay, or biochemical assay for enzyme activity.

The medium can be assayed directly, by introducing components of the assay (e.g., substrate, co-factors, antibody) into the medium (e.g., before, simultaneous with or after the cells and agent are combined). The assay can also be performed on medium which has been separated from the cells or further processed (e.g., fractionated) prior to assay. For example, convenient assays are available for enzymes, such as serine esterases (see e.g., Taub, D. D. et al., J. Immunol., 155: 3877-3888 (1995) regarding release of granule-derived serine esterases).

In another embodiment, cells expressing CX3CR1 are combined with a ligand of CX3CR1 or promoter of CX3CR1 function, an agent to be tested is added before, after or simultaneous therewith, and degranulation is assessed. Inhibition of ligand- or promoter-induced degranulation is indicative that the agent is an inhibitor of mammalian CX3CR1 function.

In a preferred embodiment, the antagonist of CX3CR1 function does not significantly inhibit the function of other chemokine receptors (e.g., CCR1, CCR2, CXCR1, CCR3). Such CX3CR1-specific antagonists can be identified by suitable methods, such as by suitable modification of the methods described herein. For example, cells which do not express CX3CR1 (CX3CR1⁻) but do express one or more other chemokine receptors (e.g., CCR2, CXCR1, CCR3) can be created or identified using suitable methods (e.g., transfection, antibody staining, western blot, RNAse protection). Such cells or cellular fractions (e.g., membranes) obtained from such cells can be used in a suitable binding assay. For example, when a cell which is CX3CR1⁻ and CCR3⁺ is chosen, the CX3CR1 antagonist can be assayed for the capacity to inhibit the binding of a suitable CCR3 ligand (e.g., RANTES, MCP-3) to the cell or cellular fraction, as described herein.

In another preferred embodiment, the antagonist of CX3CR1 function is an agent which binds to CX3CR1. Such CX3CR1-binding antagonists can be identified by suitable methods, for example, in binding assays employing a labeled (e.g., enzymatically labeled (e.g., alkaline phosphatase, horse radish peroxidase), biotinylated, radio-labeled (e.g., ³H, ¹⁴C, ¹²⁵I)) antagonist.

In another preferred embodiment, the antagonist of CX3CR1 function is an agent which can inhibit the binding of a (i.e., one or more) CX3CR1 ligand to CX3CR1 (e.g., human CX3CR1).

In particularly preferred embodiment, the antagonist of CX3CR1 function is an agent which can bind to CX3CR1 and thereby inhibit the binding of a (i.e., one or more) CX3CR1 ligand to CX3CR1 (e.g., human CX3CR1).

Antagonists of Fractalkine Function

As used herein, the term “antagonist of fractalkine function” refers to an agent (e.g., a molecule, a compound) which can inhibit a (i.e., one or more) function of fractalkine. For example, an antagonist of fractalkine function can inhibit the binding of the chemokine to one or more fractalkine receptors (e.g., CX3CR1), inhibit fractalkine-mediated cellular adhesion and/or inhibit signal transduction mediated through receptor (e.g., CX3CR1) upon fractalkine binding. Accordingly, fractalkine-mediated processes and cellular responses (e.g., proliferation, migration, adhesion, chemotactic responses, secretion or degranulation) can be inhibited with an antagonist of fractalkine function.

Preferably, the antagonist of fractalkine function is a compound which is, for example, a small organic molecule, natural product, protein (e.g., antibody, chemokine, cytokine), peptide or peptidomimetic. Antagonists of fractalkine function can be prepared and/or identified using suitable methods, such as the methods described herein or suitable modifications thereof. For example, antibodies (e.g., polyclonal antibodies, monoclonal antibodies, antigen-binding fragments of antibodies) having binding specificity for fractalkine can be prepared by immunizing a suitable host with fractalkine (e.g., isolated and/or recombinant fractalkine or a domain thereof (e.g., chemokine domain)) or with cells that express fractalkine. In one embodiment, the antagonist of fractalkine function is an antibody or antigen-binding fragment thereof having binding specificity for a mammalian fractalkine (e.g., human fractalkine).

The capacity of an agent to antagonize fractalkine function can be assessed using a suitable assay. For example, agents which inhibit the binding of fractalkine to receptor (e.g., CX3CR1) can be identified using in a receptor binding assay using labeled fractalkine where the capacity of the agent to inhibit formation of a fractalkine-receptor complex is monitored. In another example, the capacity of an agent to inhibit fractalkine-induced cellular adhesion is assessed. Cellular adherence can be monitored by methods known in the art or other suitable methods. In one embodiment, an agent to be tested can be combined with (a) non adherent cells which express mammalian fractalkine (i.e., the integral membrane form of fractalkine) or a functional variant thereof, and (b) a composition comprising a fractalkine receptor (e.g., a substrate such as a culture well coated with CX3CR1, a culture well containing adherent cells which express CX3CR1), and maintained under conditions suitable for fractalkine-receptor mediated adhesion. Labeling of cells with a fluorescent dye provides a convenient means of detecting adherent cells. Nonadherent cells can be removed (e.g., by washing) and the number of adherent cells determined. A reduction in the number of adherent cells in wells containing a test agent in comparison to suitable control wells (e.g., wells that do not contain a test agent) indicates that the agent is an antagonist of fractalkine function. The antagonist of fractalkine function can inhibit the function of transmembrane fractalkine, soluble fractalkine or other active fragments of fractalkine (e.g., fragments having chemoattractant activity).

In a preferred embodiment, the antagonist of fractalkine function does not significantly inhibit the function of other chemokines (e.g., RANTES, MIP-1α, MCP-1). Such fractalkine-specific antagonists can be identified by suitable methods, such as by suitable modification of the methods described herein. For example, cells that do not express a receptor for fractalkine (e.g., CX3CR1) but do express a receptor of another chemokine (e.g., CCR1 which binds RANTES) can be identified. The fractalkine antagonist can be assayed for the capacity to inhibit binding of a suitable ligand to such cells or cellular fractions (e.g., membranes) obtained from such cells.

In another preferred embodiment, the antagonist of fractalkine function is an agent which binds fractalkine (e.g., transmembrane fractalkine, soluble fractalkine). Such fractalkine-binding antagonists can be identified by suitable methods, for example, in binding assays employing a labeled (e.g., enzymatically labeled (e.g., alkaline phosphatase, horseradish peroxidase), biotinylated, radio-labeled (e.g., ³H, ¹⁴C, ¹²⁵I)) antagonist.

In particularly preferred embodiment, the antagonist of fractalkine function is an agent which can bind to mammalian fractalkine and thereby inhibit the binding of mammalian fractalkine (e.g., human transmembrane fractalkine, human soluble fractalkine) to a mammalian fractalkine receptor (e.g., human CX3CR1).

Therapeutic Methods

Inflammatory Arthritis

In one aspect the invention relates to a method of treating a subject having inflammatory arthritis. Treatment includes therapeutic or prophylactic treatment. Treatment, in accordance with the method, can prevent disease or reduce the severity of disease in whole or in part.

As used herein, “inflammatory arthritis” refers to those diseases of joints where the immune system is causing or exacerbating inflammation in the joint, and includes rheumatoid arthritis and spondyloarthropathies, such as ankylosing spondylitis, reactive arthritis, Reiter's syndrome, psoriatic arthritis, psoriatic spondylitis, enteropathic arthritis, enteropathic spondylitis, juvenile-onset spondyloarthropathy and undifferentiated spondyloarthropathy. Inflammatory arthritis is generally characterized by infiltration of the synovial tissue and/or synovial fluid by leukocytes.

In one embodiment, the method of treating inflammatory arthritis comprises administering an effective amount of an (i.e., one or more) antagonist of CX3CR1 function to a subject in need thereof.

In a preferred embodiment, the invention provides a method of treating (including therapeutic or prophylactic treatment) rheumatoid arthritis, comprising administering an effective amount of an antagonist of CX3CR1 function to a subject in need thereof.

In particular embodiments, the antagonist of CX3CR1 function is selected from the group of molecules which can inhibit one or more functions of CX3CR1, for example, certain small organic molecules, natural products, peptides, peptidomimetics and proteins, wherein said proteins are not chemokines or mutants or analogues thereof.

In other embodiments, the invention provides a method for treating (preventing or reducing the severity of) inflammatory arthritis comprising administering an effective amount of an antagonist of CX3CR1 function and an effective amount of an (i.e., one or more) additional therapeutic agent to a subject in need thereof. The therapeutic benefit of an antagonist of CX3CR1 function and certain other therapeutic agents can be additive or synergistic when co-administered, thereby providing a highly efficacious treatment.

The invention also relates to a method for treating a subject having inflammatory arthritis comprising administering an effective amount of an (i.e., one or more) antagonist of fractalkine function to a subject in need thereof.

In a preferred embodiment, the invention provides a method of treating (including therapeutic or prophylactic treatment) rheumatoid arthritis, comprising administering an effective amount of an antagonist of fractalkine function to a subject in need thereof.

In particular embodiments, the antagonist of fractalkine function is selected from the group of molecules which can inhibit one or more functions of fractalkine, for example, certain small organic molecules, natural products, peptides, peptidomimetics and proteins, wherein said proteins are not chemokines or mutants or analogues thereof.

In other embodiments, the invention provides a method for treating (preventing or reducing the severity of) inflammatory arthritis comprising administering an effective amount of an antagonist of fractalkine function and an effective amount of an (i.e., one or more) additional therapeutic agent to a subject in need thereof. The therapeutic benefit of an antagonist of fractalkine function and certain other therapeutic agents can be additive or synergistic when co-administered, thereby providing a highly efficacious treatment.

Therapeutic agents suitable for co-administration with an antagonist of CX3CR1 function and/or an antagonist of fractalkine function include, for example, antiviral agents (e.g., acyclovir, ganciclovir, famciclovir, penciclovir, valacyclovir, vidarabine, foscarnet, indinavir), antibacterial agents (e.g., antibiotics (e.g., erythromycin, penicillin, tetracycline, ciprofloxacin, norfloxacin, flurazolidone, azithromycin, chloramphenicol), sulfonamides, quinalones). Preferred therapeutic agents for co-administration include, for example, methotrexate, anti-inflammatory agents (e.g., nonsteroidal anti-inflammatory agents, such as aspirin, ibuprofen, naproxen, lysofylline, inhibitors of cyclooxygenase-2), cytokines (e.g., TGFβ), immunosuppressive agents, such as, calcineurin inhibitors (e.g., cyclosporin A, FK-506), IL-2 signal transduction inhibitors (e.g., rapamycin), glucocorticoids (e.g., prednisone, dexamethasone, methylprednisolone), nucleic acid synthesis inhibitors (e.g., azathioprine, mercaptopurine, mycophenolic acid), and antibodies to lymphocytes and antigen-binding fragments thereof (e.g., OKT3, anti-L2 receptor), disease modifying anti-rheumatic agents (e.g., D-penicillamine, sulfasalazine, chloroquine, hydroxychloroquine) and antibodies, such as antibodies that bind chemokines, cytokines (e.g., anti-TNFα) or cell adhesion molecules (e.g., anti-CD11/CD18).

The particular co-therapeutic agent selected for administration with an antagonist of CX3CR1 and/or an antagonist of fractalkine function will depend on the type and severity of inflammatory arthritis being treated as well as the characteristics of the individual, such as general health, age, sex, body weight and tolerance to drugs. For example, in one embodiment, an antagonist of CX3CR1 function can be administered together with oral prednisone to treat rheumatoid arthritis. In another embodiment, an antagonist of fractalkine function can be administered together with a nonsteroidal anti-inflammatory agent (e.g., aspirin). The skilled artisan will be able to determine the preferred co-therapeutic agent based upon these considerations and other factors.

The invention further relates to an antagonist of CX3CR1 function and/or an antagonist of fractalkine function for use in therapy (including prophylaxis) or diagnosis, for example, as described herein, and to the use of such an antagonist for the manufacture of a medicament for the treatment of inflammatory arthritis (e.g., rheumatoid arthritis). The invention also relates to a medicament for the treatment of inflammatory arthritis (e.g., rheumatoid arthritis) wherein said medicament comprises an antagonist of CX3CR1 function and/or an antagonist of fractalkine function.

The invention also relates to a method for treating a subject having inflammatory arthritis comprising administering an effective amount of an (i.e., one or more) antagonist of fractalkine function and an antagonist of CX3CR1 function to a subject in need thereof.

Angiogenesis

In another aspect, the invention relates to a method of inhibiting angiogenesis (e.g., fractalkine-induced angiogenesis) in a subject in need thereof. Treatment includes therapeutic or prophylactic treatment. According to the method, angiogenesis (e.g., pathogenic neovascularization) can be inhibited in whole or in part.

As used herein, “pathogenic neovascularization” refers to the proliferation and/or formation of blood vessels in tissue not normally containing them, to the proliferation of blood vessels of a different kind than are normally found in a tissue or to the proliferation of blood vessels beyond the amount typically present in a tissue (hypervascularization). Pathogenic neovascularization includes angiogenesis associated with cancers (e.g., tumor formation and growth and/or metastasis), retinopathy (e.g., retinopathy of prematurity, diabetic retinopathy), retinal vein occlusion, macular degeneration (e.g., age-related macular degeneration), hemangiomas, inflammatory arthritis (e.g., rheumatoid arthritis) and psoriasis. Accordingly, the present invention provides a method of treating such diseases by administering an effective amount of an antagonist of fractalkine function and/or an antagonist of CX3CR1 function to a subject in need thereof.

In one embodiment, the method of inhibiting (including therapeutic or prophylactic treatment) angiogenesis comprises administering an effective amount of an (i.e., one or more) antagonist of CX3CR1 function to a subject in need thereof.

In particular embodiments, the antagonist of CX3CR1 function is selected from the group of molecules which can inhibit one or more functions of CX3CR1, for example, certain small organic molecules, natural products, peptides, peptidomimetics and proteins, wherein said proteins are not chemokines or mutants or analogues thereof.

In other embodiments, the invention provides a method for inhibiting angiogenesis comprising administering an effective amount of an antagonist of CX3CR1 function and an effective amount of an (i.e., one or more) additional therapeutic agent to a subject in need thereof. The therapeutic benefit of an antagonist of CX3CR1 function and certain other therapeutic agents can be additive or synergistic when co-administered, thereby providing a highly efficacious treatment.

The invention also relates to a method of inhibiting (including therapeutic or prophylactic treatment) angiogenesis comprising administering an effective amount of an (i.e., one or more) antagonist of fractalkine function to a subject in need thereof.

In particular embodiments, the antagonist of fractalkine function is selected from the group of molecules which can inhibit one or more functions of fractalkine (e.g., receptor binding), for example, certain small organic molecules, natural products, peptides, peptidomimetics and proteins, wherein said proteins are not chemokines or mutants or analogues thereof.

In other embodiments, the invention provides a method for inhibiting angiogenesis comprising administering an effective amount of an antagonist of fractalkine function and an effective amount of an (i.e., one or more) additional therapeutic agent to a subject in need thereof. The therapeutic benefit of an antagonist of fractalkine function and certain other therapeutic agents can be additive or synergistic when co-administered, thereby providing a highly efficacious treatment.

Preferred agents for co-administration when inhibition of angiogenesis is desired include agents which inhibit angiogenesis or inhibit the activity of angiogenic factors, such as thalidomide, Angiostatin™, Endostatin™, 2-methoxyestradiol, antagonists of the IL-8 receptor (see, U.S. Pat. No. 6,105,908) and the like. Additional therapeutic agents suitable for co-administration with an antagonist of CX3CR1 function and/or an antagonist of fractalkine function when inhibition of angiogenesis is desired include, for example, antiviral agents (e.g., acyclovir, ganciclovir, famciclovir, penciclovir, valacyclovir, vidarabine, foscarnet, indinavir), antibacterial agents (e.g., antibiotics (e.g., erythromycin, penicillin, tetracycline, ciprofloxacin, norfloxacin, flurazolidone, azithromycin, chloramphenicol), sulfonamides, quinalones), methotrexate, anti-inflammatory agents (e.g., nonsteroidal anti-inflammatory agents, such as aspirin, ibuprofen, naproxen, lysofylline, inhibitors of cyclooxygenase-2), cytokines (e.g., TGFβ), immunosuppressive agents, such as, calcineurin inhibitors (e.g., cyclosporin A, FK-506), IL-2 signal transduction inhibitors (e.g., rapamycin), glucocorticoids (e.g., prednisone, dexamethasone, methylprednisolone), nucleic acid synthesis inhibitors (e.g., azathioprine, mercaptopurine, mycophenolic acid), and antibodies to lymphocytes and antigen-binding fragments thereof (e.g., OKT3, anti-IL2 receptor), disease modifying anti-rheumatic agents (e.g., D-penicillamine, sulfasalazine, chloroquine, hydroxychloroquine) and antibodies, such as antibodies that bind chemokines, cytokines (e.g., anti-TNFα) or cell adhesion molecules (e.g., anti-CD11/CD18).

The invention further relates to an antagonist of CX3CR1 function and/or an antagonist of fractalkine function for use in therapy (including prophylaxis), for example, to inhibit angiogenesis as described herein, and to the use of such an antagonist for the manufacture of a medicament for inhibiting angiogenesis (e.g., pathogenic neovascularization). The invention also relates to a medicament for inhibiting angiogenesis (e.g., pathogenic neovascularization) wherein said medicament comprises an antagonist of CX3CR1 function and/or an antagonist of fractalkine function.

The invention also relates to a method of inhibiting (including therapeutic or prophylactic treatment) angiogenesis comprising administering an effective amount of an (i.e., one or more) antagonist of fractalkine function and an antagonist of CX3CR1 function to a subject in need thereof.

Modes of Administration

A “subject” is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

An “effective amount” of an antagonist of CX3CR1 function or an antagonist of fractalkine function is an amount sufficient to achieve a desired therapeutic and/or prophylactic effect, such as an amount sufficient to inhibit joint inflammation, to inhibit joint pain or to inhibit formation of blood vessels. For example, an effective amount of an antagonist of CX3CR1 function is an amount sufficient to inhibit a (i.e., one or more) function of CX3CR1 (e.g., CX3CR1 ligand-induced leukocyte migration, CX3CR1 ligand-induced integrin activation, CX3CR1 ligand-induced transient increase in the concentration of intracellular free calcium [Ca²⁺]₁ and/or CX3CR1 ligand-induced secretion (e.g. degranulation) of proinflammatory mediators), and thereby, inhibit joint inflammation, joint pain or formation of blood vessels. An effective amount of an antagonist of fractalkine function can be an amount sufficient to inhibit a (i.e., one or more) function of fractalkine (e.g., binding to a fractalkine receptor (e.g., CX3CR1), fractalkine receptor-mediated leukocyte migration, fractalkine receptor-mediated integrin activation, fractalkine receptor-induced transient increase in the concentration of intracellular free calcium [Ca²⁺]₁ and/or fractalkine receptor-induced secretion (e.g. degranulation) of proinflammatory mediators), and thereby, inhibit joint inflammation, joint pain or formation of blood vessels. An “effective amount” of an additional therapeutic agent (e.g., immunosuppressive agent) is an amount sufficient to achieve a desired therapeutic and/or prophylactic effect (e.g., immunosuppression).

The amount of agent (e.g., CX3CR1 antagonist, fractalkine antagonist, additional therapeutic agent) administered to the individual will depend on the characteristics of the individual, such as general health, age, sex, body weight and tolerance to drugs as well as the degree, severity and type of disease and desired therapeutic effect. The skilled artisan will be able to determine appropriate dosages depending on these and other factors. Typically, an effective amount can range from about 0.01 mg per day to about 100 mg per day for an adult. Preferably, the dosage ranges from about 1 mg per day to about 100 mg per day.

The agent (e.g., CX3CR1 antagonist, fractalkine antagonist, additional therapeutic agent) can be administered by any suitable route, including, for example, orally (e.g., in capsules, suspensions or tablets) or by parenteral administration. Parenteral administration can include, for example, intramuscular, intravenous, intraarticular, intrathecal, subcutaneous, or intraperitoneal administration. The agent (e.g., CX3CR1 antagonist, fractalkine antagonist, additional therapeutic agent) can also be administered orally (e.g., dietary), transdermally, topically, by inhalation (e.g., intrabronchial, intranasal, oral inhalation or intranasal drops) or rectally. Administration can be local or systemic as indicated. The preferred mode of administration can vary depending upon the particular agent (e.g., CX3CR1 antagonist, fractalkine antagonist, additional therapeutic agent) chosen, however, oral or parenteral administration is generally preferred.

The agent (e.g., CX3CR1 antagonist, fractalkine antagonist, additional therapeutic agent) can be administered as a neutral compound or as a salt. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

When co-administration of an antagonist (i.e., an antagonist of CX3CR1 function and/or an antagonist of fractalkine function, as described herein) and an additional therapeutic agent is indicated or desired for treating inflammatory arthritis, the antagonist (i.e., antagonist of CX3CR1 function and/or antagonist of fractalkine function) can be administered before, concurrently with or after administration of the additional therapeutic agent. When the antagonist and additional therapeutic agent are administered at different times, they are preferably administered within a suitable time period to provide substantial overlap of the pharmacological activity of the agents. The skilled artisan will be able to determine the appropriate timing for co-administration of antagonists (i.e., antagonist of CX3CR1 function and/or antagonist of fractalkine function) and an additional therapeutic agent depending on the particular agents selected and other factors.

The antagonist (i.e., an antagonist of CX3CR1 function and/or an antagonist of fractalkine function, as described herein) can be administered to the individual as part of a pharmaceutical or physiological composition for treating inflammatory arthritis comprising or inhibiting angiogenesis. Such a composition can comprise and antagonist (i.e., an antagonist of CX3CR1 function and/or an antagonist of fractalkine function, as described herein) and a physiologically acceptable carrier. Pharmaceutical compositions for co-therapy can further comprise one or more additional therapeutic agents. Alternatively, an antagonist (i.e., an antagonist of CX3CR1 function and/or an antagonist of fractalkine function, as described herein) and an additional therapeutic agent can be components of separate pharmaceutical compositions which can be mixed together prior to administration or administered separately. Formulation will vary according to the route of administration selected (e.g., solution, emulsion, capsule). Suitable pharmaceutical carriers can contain inert ingredients which do not interact with the antagonist of CX3CR1 function and/or additional therapeutic agent. Standard pharmaceutical formulation techniques can be employed, such as those described in Remington's Pharmaceutical Sciences, Mack Publishing Company, Easton, Pa. Suitable physiological carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

Diagnostic Applications

As described herein, rheumatoid arthritis can be distinguished from other arthropathies bases on the amount of soluble fractalkine contained in synovial fluid obtained from diseased joints. Accordingly, another aspect of the invention relates to a method for diagnosing rheumatoid arthritis. The method comprises a) determining the amount of soluble fractalkine contained in a sample of synovial fluid obtained from a subject suspected of having rheumatoid arthritis, and b) comparing the amount determined in a) with a suitable control, wherein an elevated amount of soluble fractalkine relative to the control is indicative of rheumatoid arthritis. Samples of synovial fluid can be obtained using suitable methods, such as arthrocentesis. The amount of soluble fractalkine in synovial fluid can be determined directly or indirectly using any suitable method. For example, the amount can be determined by measuring the absolute quantity of soluble fractalkine in the sample or by determining if the amount of soluble fractalkine in a sample exceeds a predetermined threshold level (e.g., 1 ng/ml, 500 pg/ml, 50 pg/ml, 5 pg/ml). Suitable methods for determining the amount of soluble fractalkine in a sample include immunological and immunochemical methods such as immunosorbent assays, including enzyme-linked immunosorbent assays (ELISA), radioimmunoassay (RIA), chemiluminescence assays, immuno-blot (e.g., western blot), immunocytochemistry and immunohistology. Generally, a sample and antibody or antigen-binding fragment thereof are combined under conditions suitable for the formation of a complex between fractalkine and said antibody or fragment, and the formation of said complex is assessed (directly or indirectly).

Suitable controls for comparison include the measured amount of soluble fractalkine contained in synovial fluid obtained from an individual who does not have rheumatoid arthritis and the measured amount of soluble fractalkine contained in serum obtained from a healthy donor or a donor with arthritis. In one embodiment, the amount of soluble fractalkine contained in synovial fluid obtained from an individual suspected of having rheumatoid arthritis is compared to the average amount of soluble fractalkine contained in synovial fluids obtained from a population (e.g. 10, 20, 50, 100 or more individuals) in which each individual has an arthritis that is not rheumatoid arthritis or in which each individual is healthy. In another embodiment, the amount of soluble fractalkine contained in synovial fluid obtained from an individual suspected of having rheumatoid arthritis is significantly higher than a suitable control as determined by a suitable statistical test, such as the Student's t test.

The method of diagnosing rheumatoid arthritis can include one or more additional clinical or laboratory test. Several tests which can be used to provide a further indication of rheumatoid arthritis are known in the art. For example, a high titer of rheumatoid factor, increased erythrocyte sedimentation rate, increased ceruloplasmin levels, increased C-reactive protein levels, further synovial fluid analyses (e.g., decreased viscosity, increased protein content, decreased glucose levels, high white blood cell count) and/or radiographic evidence of cartilage destruction can provide a further indication that the subject has rheumatoid arthritis.

The amount of soluble fractalkine contained in synovial fluid obtained from an individual with rheumatoid arthritis can be of prognostic significance. For example, the amount of soluble fractalkine in the synovial fluid of an individual with rheumatoid arthritis can correlate with and be a predictive indicator of disease severity, progression and/or the appearance of extra-articular manifestations (e.g., rheumatoid nodules, rheumatoid vasculitis, osteoporosis, Felty's syndrome, pleural disease, interstitial fibrosis, pleuropulmonary nodules, pneumonitis, arteritis).

A course of therapy for treating rheumatoid arthritis can be monitored by determining the amount of soluble fractalkine contained in synovial fluid obtained from an individual undergoing such therapy. In one example, the amount of soluble fractalkine in a sample of synovial fluid obtained prior to starting therapy is determined. After therapy has begun, another sample of synovial fluid is obtained and the amount of soluble fractalkine contained in the sample is determined. A decrease in the amount of fractalkine in synovial fluid samples obtained after the start of therapy relative to the amount in samples obtained prior to therapy can indicate that the therapy is efficacious. The amount of soluble fractalkine contained in samples of synovial fluid obtained at predetermined intervals (e.g., weekly, monthly) can be determined to monitor the efficacy of therapeutic interventions.

In another aspect of the invention, rheumatoid arthritis can be diagnosed based on fractalkine (e.g., non-soluble fractalkine) expression. As described herein, fkn expression and early morning stiffness (EMS) correlate positively with RA SF CD14+ cells. Thus, in one embodiment, the method of diagnosing rheumatoid arthritis comprises a) determining fkn expression on CD14+ cells contained in a sample of synovial fluid obtained from a subject suspected of having rheumatoid arthritis, and b) comparing the amount determined in a) with a suitable control, wherein an elevated amount of fkn is indicative of rheumatoid arthritis.

In another aspect of the invention, rheumatoid arthritis can be diagnosed based on CX3CR1 expression. As described herein, CX3CR1 expression and the age of the patient correlate positively with RA PB CD3+ cells. Moreover, CX3CR1 expression and patient swollen joint count (SJC) correlate positively with RA SF CD3+ cells. Thus, in one embodiment, the method of diagnosing rheumatoid arthritis comprises a) determining CX3CR1 expression in a sample of peripheral blood obtained from a subject suspected of having rheumatoid arthritis, and b) comparing the amount determined in a) with a suitable control, wherein an elevated amount of CX3CR1 is indicative of rheumatoid arthritis. In a particular embodiment, the method of diagnosing rheumatoid arthritis comprises determining CX3CR1 expression on CD3+ cells contained in a sample of peripheral blood obtained from a subject suspected of having rheumatoid arthritis.

In another embodiment, the method of diagnosing rheumatoid arthritis comprises a) determining CX3CR1 expression in a sample of synovial fluid obtained from a subject suspected of having rheumatoid arthritis, and b) comparing the amount determined in a) with a suitable control, wherein an elevated amount of CX3CR1 is indicative of rheumatoid arthritis. In a particular embodiment, the method of diagnosing rheumatoid arthritis comprises determining CX3CR1 expression on CD3+ cells contained in a sample of synovial fluid obtained from a subject suspected of having rheumatoid arthritis,

The amount of CX3CR1 in a biological sample (e.g., peripheral blood, synovial fluid) or a fraction of a biological sample (e.g., CD3+ cells, CD14+ cells) can be determined directly or indirectly using any suitable method. For example, CD3+ cells can be isolated and identified using flow cytometry or other suitable methods. The amount of CX3CR1 on CD3+ cells can be determined by measuring the absolute quantity of CX3CR1 on CD3+ cells or by determining if the amount of CX3CR1 on CD3+ cells exceeds a predetermined threshold level (e.g., 1 ng/ml, 500 pg/ml, 50 pg/ml, 5 pg/ml). Suitable methods for determining the amount of CX3CR1 in a sample containing CD3+ cells include immunological and immunochemical methods such as immunosorbent assays, including enzyme-linked immunosorbent assays (ELISA), radioimmunoassay (RIA), chemiluminescence assays, immuno-blot (e.g., western blot), immunocytochemistry, immunohistology and flow cytometry. Generally, a sample containing CD3+ cells and antibody or antigen-binding fragment thereof are combined under conditions suitable for the formation of a complex between CX3CR1 and said antibody or fragment, and the formation of said complex is assessed (directly or indirectly).

Suitable controls for comparison include the measured amount of CX3CR1 contained on CD3+ cells from peripheral blood or synovial fluid obtained from an individual who does not have rheumatoid arthritis. In another embodiment, the amount of CX3CR1 on CD3+ cells contained in peripheral blood or synovial fluid obtained from an individual suspected of having rheumatoid arthritis is significantly higher than a suitable control as determined by a suitable statistical test, such as the Student's t test.

The present invention will now be illustrated by the following Examples, which are not intended to be limiting in any way.

### EXAMPLE 1

Materials and Methods

Animals

Female Lewis rats were obtained from Harlan (Indianapolis, Ind.) maintained under specific pathogen-free conditions and provided with food and water ad libitum.

Induction of Adjuvant Induced Arthritis (AIA)

The rat AIA model was performed as described previously (Halloran, M. et al., J. Immunol., 65:7492 (1999); Halloran, M. et al., Arthritis Rheum., 39:810 (1996)). Briefly, female Lewis rats (100 g) were injected subcutaneously with 0.3 ml lyophilized Mycobacterium butyricum (5 mg/ml) (Difco Laboratories, Detroit, Mich.) at the base of the tail. AIA affects mostly the hind limbs. Therefore, the degree of arthritis, indicated by joint swelling, was quantified by measuring two perpendicular diameters of the ankles using calipers (Lange Caliper, Cambridge Scientific Industries, Cambridge Mass.). Joint circumference was measured on days 0, 4, 7, 11, 18, 25, 41, and 47 (n=3 rats per time point) post-adjuvant injection and calculated using a geometric formula

For immunohistochemical studies, rats were allowed to develop arthritis and sacrificed on day 0 or 4, 7, 11, 18, 25, 41, 47 or 54 days following administration of adjuvant. The day of tail injection was considered day 0. Ankles for tissue sectioning were removed. The skin was removed, and the isolated tissue was embedded in OCT Media (Miles, Elkhart, Ind.) and frozen at −80° C. until sectioning. Sections (7 μm) were cut using a D-profile knife suitable for bone cutting (Leica, Nussloch, Germany). The tissue sections were placed on glass slides and stored at −80° C. until used in immunohistochemistry.

Human Samples

Synovial fluid (SF) samples were obtained during arthrocentesis from patients with rheumatoid arthritis (RA), osteoarthritis (OA), and other diseases including juvenile rheumatoid arthritis (JRA), psoriatic arthritis (PSA), polyarthritis (PA), spondyloarthropathy (ANK), inflammatory myopathy (IM), and gout. Peripheral blood (PB) sera was collected from 12 patients with arthritic diseases (RA, OA, JRA, PSA, PA and gout) and 10 healthy donors. Synovial tissues (ST) from RA patients were obtained from patients undergoing total joint replacement who met the American College of Rheumatology criteria for RA. Normal STs were obtained from fresh autopsies or from amputations. All specimens were obtained with Institutional Review Board approval. Clinical data that were collected included age of the patient, duration of disease, erythrocyte sedimentation rate (ESR), early morning stiffness (EMS) and swollen joint count (SJC).

Immunohistochemistry

Frozen tissue (rat AIA and patient RA ST) sections (7 μm) were cut and immunoperoxidase stained with an avidin-biotin technique (Vector Laboratories, Burlingame, Calif.) with all subsequent incubations being performed at 37° C. in a humidified chamber. Slides were fixed in cold acetone for 20 minutes or in 2% paraformaldehyde for 10 minutes at 4° C., and then treated with 3% peroxidase in 0.1 M Tris for 5 min to block endogenous peroxidase activity. Tissues were blocked with 3% horse serum (in PBS) for 1 hour, then incubated with mouse anti-human fkn IgM (LeukoSite, now Millennium Pharmaceuticals Inc., Cambridge, Mass.), purified mouse IgM (Coulter, Miami, Fla.), mouse anti-human CX3CR1 IgG (LeukoSite, now Millennium Pharmaceuticals Inc., Cambridge, Mass.), or purified mouse IgG (Coulter, Miami, Fla.) for an additional hour. Tissue was washed twice in PBS, and a 1:200 dilution (in PBS) of biotinylated anti-mouse antibody (Vector Laboratories, Burlington, Mass.) was added to the tissue sections and the tissue sections were incubated for an additional 20 minutes. After a final washing (2× in PBS), slides were developed with a diaminobenzidine tetrahydrochloride substrate (Vector Laboratories) for 2 min at room temperature (RT), rinsed in tap water, counter-stained with Harris' Hematoxylin, and dipped in saturated lithium carbonate solution for bluing. For rat AIA kinetic studies, serial tissue sections were examined by a blinded pathologist to determine the percentage of each cell type expressing immunoreactive fkn or CX3CR1 for each time point. Various ST cell types were identified including macrophages, lymphocytes, fibroblasts, endothelial cells, and dendritic cells by immunohistochemical staining reactions and/or morphological features. Human macrophages were identified morphologically and with anti-LeuM5 (Becton Dickinson, San Jose, Calif.) and were CD68⁺ (CD68 stain is clone EBM-11, Dako, IgG1, Carpinteria, Calif.) in serial sections. Endothelium was verified using anti-von Willebrand's factor (Dako, Carpinteria, Calif.). Rat dendritic cells were identified with a murine anti-rat dendritic cell marker (OX-62 IgG, PharMingen, San Diego, Calif.). Immunostaining for rat AIA kinetic studies was graded by a frequency of staining scale (0-100%), where 0% indicated no staining and 100% showed that all the cells were immunoreactive for each of the ST components.

Flow Cytometry

Leukocytes were harvested from RA SFs by passage through a 40 μm mesh nylon filter to remove cellular debris. SF samples were centrifuged to pellet the cells and the pelleted cells were washed twice with FACS buffer (PBS+1% fetal bovine serum (FBS)). Cells were counted, re-suspended at 1×10⁷ cells/ml in blocking buffer (1% BSA, 30% goat serum, and 0.1% NaN₃ in PBS) and incubated for 15 min at 4° C. One million SF cells in 100 μl FACS buffer (or 1 ml of whole blood for PB samples) were incubated with primary Abs (mAb mouse anti-human fractalkine IgM, LeukoSite Inc., Cambridge, Mass.), or nonspecific mouse IgM (Sigma Chemical Company, St. Louis, Mo.) at a final concentration of 10 μg/ml for 30 min at 4° C. For CX3CR1 studies, leukocytes were incubated with either mAb mouse anti-human fkn IgG1 (LeukoSite Inc., Cambridge, Mass.) or nonspecific (ns) mouse IgG1 (Sigma Chemical Company, St. Louis, Mo.) then washed with FACS buffer and incubated with diluted (1:100 in PBS) goat anti-mouse IgM (for fkn) or goat anti-mouse IgG (for CX3CR1) R-Phycoerythrin (PE) antibody (Jackson Immunoresearch Laboratory, West Grove, Pa.) for 30 min at 4° C. Cells were washed once more with FACS buffer, and incubated with mouse serum for 10 minutes at RT. FITC-conjugated anti-CD3 (detects T cells) or FITC-conjugated anti-CD14 (detects monocytes) (PharMingen, San Diego, Calif.) were added to the cells and the cells were incubated for 30 minutes at 4° C. Two ml of 1×Becton Dickinson (Bedford, Mass.) lysing reagent was added to the whole blood samples. The resulting mixture was incubated in the dark at RT for 10 minutes to allow for red blood cell lysis. Samples were washed (2×) with FACS buffer, fixed with 500 μL 1% formaldehyde (in PBS), then assayed in a Coulter EPICS XL-MCL instrument (Beckman Coulter Inc., Fullerton, Calif.). Results were expressed as the percentage of cells staining with control antibody with the background subtracted.

Depletion of Rheumatoid Factor (RF) from Sera and SF

To avoid any possible confounding effects by RF on assays, RF was immunodepleted from sera and SFs using anti-IgM antibodies coupled to agarose beads (Sigma Chemical Co., St. Louis, Mo.). One half ml of bead slurry was washed 3× with PBS (the fluid layer being aspirated off), and dry beads were mixed with 1.0 ml SF or sera (diluted 1:2 in PBS) and incubated overnight at 4° C. with constant shaking. Then samples were spun down (2000 rpm for 2 minutes), the supernatant was collected at a final dilution of 1:2. Removal of RF (IgM) was continued by randomly choosing five RA SF samples and measuring RF levels before and after immunodepletion (rheumatoid factor ELISA kit, RDI, Flanders, N.J.). Before immunodepletion, RF levels ranged from 5 to 300 international units/ml. After immunodepletion, all samples had RF levels below the detection limit of the assay (0.031 international units/ml). Samples immunodepleted of RF were used in ELISA and in chemotaxis studies.

Measurement of sfkn

Unless otherwise indicated, reagents were obtained from R&D Systems, Minneapolis, Minn. Human sfkn was measured in SF and sera by ELISA. 96 well polystyrene plates were coated overnight at 4° C. with (0.05 ml/well) 4 μg/ml purified goat anti-human fkn IgG. The plates were washed with washing buffer (0.05% PBS-Tween), and then blocked with 0.2 ml/well of 1% BSA/5% sucrose dissolved in PBS for two hours at RT. Recombinant human fkn or test supernatants were added to triplicate wells (0.05 ml/well), and the plates were incubated at 4° C. overnight. The plates were washed 3 times and biotinylated goat anti-human fkn antibody (diluted 1:23 in 10% FBS/PBS) was added to each well (0.05 ml/well). The plates were then incubated for 45 minutes at RT. Following the incubation, the plates were washed 3 times, streptavidin-peroxidase (Pharmingen, San Diego, Calif., diluted 1:10,000 in 10% FBS/PBS) was added to the wells (0.1 ml/well), and the plates were incubated at RT for 1 hour. The plates were again washed 3 times and tetramethylbenzidine (Sigma Chemical Co., St. Louis Mo.) substrate diluted in a citrate/phosphate buffer was added to each well (0.2 ml/well) and the plates were incubated at room temperature to allow color to develop. Reactions were stopped by adding 2M H₂SO₄ (50 μl/well) to the wells and color was measured using an ELISA reader. Assay sensitivity was 500 pg/ml, and goat anti-human fkn antibody demonstrated less than 5% cross reactivity with recombinant human (rh) 6Ckine (rh6Ckine), recombinant murine (rm) 6Ckine (rm6Ckine), and less than 2% cross reactivity with rh monocyte chemotactic protein-1 (rhMCP-1), rh monocyte chemotactic protein-2 (rhMCP-2), rm monocyte chemotactic protein-3 (rmMARC), rh eotaxin, and rm eotaxin.

Isolation of Human Monocytes and Chemotaxis

Peripheral blood (PB) was collected in heparinized tubes from normal adult donors. After density gradient centrifugation on an Accu-Prep gradient at 400×g for 30 minutes RT, the buffy coat was collected and mononuclear cells were purified under sterile conditions. The collected mononuclear cells were washed twice with PBS, and re-suspended at 2.5×10⁶ cells/ml in Hanks balanced salt solution (HBSS) with calcium and magnesium (Life Technologies, Bethesda, Md.). Mononuclear cell viability was >98% (purity>99%) as determined by trypan blue exclusion. Monocyte separation was done as described previously (Denholm, E. et al., J. Immunol. Methods, 144:247 (1991)). Briefly, 4 ml of mononuclear cells were mixed with 8 ml of isolation buffer (1.65 ml 10×HBSS in 10 ml Percoll, pH 7.0) in a 15 ml siliconized tube. After centrifugation (400×g for 25 min at room temperature), monocytes were collected from the top layer of solution (top 5 mm). Monocytes were 90% pure and viability was >98% by trypan blue exclusion.

Monocyte chemotaxis assays were performed using 48 well chemotaxis chambers (Neuroprobe, Cabinjohn, Md.) with a 5 mm polyvinylpyrolidone-free polycarbonate filter (Poretics Corp., Livemore, Calif.) as previously described (Volin, M. et al, Clin. Immunol. Immunopathol., 89:44 (1998)). 25 μl of stimulant or buffer was added to the bottom wells of the chambers. A 5 μm membrane was placed in the assembly, and 40 μl of monocytes (2.5×10⁶ cells/ml) were placed in the top wells. The chemotaxis chamber was incubated for one hour in a 5% CO₂ atmosphere at 37° C. The filters were removed, the membrane were fixed in methanol and stained with Diff-Quik (Baxter Diagnosis, Chicago, Ill.). Assays were performed in quadruplicate with three high-power microscope (400×) fields counted in each replicate well. Results were expressed as number (#) monocytes migrated per high-power field. For fkn neutralization studies, SFs were pre-incubated with either goat anti-human fln IgG antibody (R&D Systems, Minneapolis, Minn.) or an equivalent amount of a corresponding control antibody (non-specific goat IgG, Coulter, Miami, Fla.) for 1 hour at 37° C. Neutralized SFs were assayed for chemotactic activity for normal PB monocytes. All chemotaxis assays included HBSS (negative control) and N-formyl-methionyl-leucyl-phenylalanine (fMLF) (positive control).

mRNA Isolation from Rat Ankles

Inflamed rat tissue was snap-frozen in liquid nitrogen and total cellular RNA was extracted using the Ultraspec RNA isolation system (Biotec Laboratories, Inc., Houston, Tex.). Frozen rat tissue (roughly 100 mg) was homogenized in 1 ml of homogenization buffer (Ultraspec RNA buffer, Biotec Laboratories, Inc.) and the homogenate was added to 0.2 volumes of chloroform. The sample was centrifuged at 12,000 g at 4° C. for 15 minutes and RNA was precipitated from the aqueous phase by adding 1 volume of sample to an equal volume of isopropanol. The precipitated RNA was washed twice in ice cold 75% ethanol. The RNA pellet was dissolved in diethyl pyrocarbonate (DEPC) water and RNA concentrations were determined spectrophotometrically prior to storage at −80° C. Yields were routinely greater than 1 mg.

Reverse Transcriptase Polymerase Chain Reaction (RT-PCR) Detection of fkn and CX3CR1 mRNA

All reagents were from Life Technologies (Grand Island, N.Y.) unless otherwise noted. The isolated RNA was first reverse transcribed (RT) to cDNA as follows. RNA (5 μg) was added to 1 μl of random primer (from a stock of 50 ng/μl) and brought up to a total of 10 μl with DEPC water. The sample was heated to 65° C. for 5 minutes in order to denature the primer and RNA. After heating, 10 μl of master mix was added to the RNA primer mix. The master mix consisted of 4 μl of 5×cDNA synthesis buffer, 0.1 M dithiothreitol (DDT), 1 μl of RN_{ASE}OUT (40 U/μl), 1 μl of DEPC water and 1 μl of Thermoscript RT (15 units/μl). The samples were transferred to a thermocycler and incubated at 60° C. for 60 minutes. The reaction was terminated by incubating the samples at 85° C. for 5 minutes. Target cDNA was amplified in a polymerase chain reaction (PCR) using 2 μl of cDNA, 5 μl of 10×High fidelity PCR buffer, 2 μl of 50 mM MgSO₄, 1 μl of 10 mM dNTP mix, 1 μl of 10 mM forward primer, 1 μl of 10 mM reverse primer, 0.2 μl of Platinum Taq High Fidelity and 39.8 μl of DEPC-treated water, in a thin-walled PCR tube. Amplification was performed in a thermocycler as follows: 2 minutes at 94° C. followed by 40 cycles of 15 seconds at 94° C., 15 seconds at 55° C. and 1 minute at 68° C. The sequences of the synthetic oligonucleotide primers that were used were as follows: rat fkn forward primer; 5′ gaattcctggcgggtcagcacctcggcata 3′ (SEQ ID NO:5), rat fkn reverse primer; 5′ aagcttttacagggcagcggtctggtggt 3′ (SEQ ID NO:6), rat CX3CR1 forward primer; 5′ agctgctcaggacctcaccat 3′ (SEQ ID NO:7), rat CX3CR1 reverse primer; 5′ gttgtggaggccctcatggctgat 3′ (SEQ ID NO:8), rat GAPDH forward primer; 5′ gaacatcatccctgcatcca 3′ (SEQ ID NO:9) and rat GAPDH reverse primer; 5′ ccagtgagcttcccgttca 3′ (SEQ ID NO:10) (see Nishiyori et al., FEBS Lett. 429: 167-172 (1998), Nishiyori et al., FEBS Lett. 429: 167-172 (1998) and Harrison et al., Brain Res. Mol. Brain Res. 75:143-149 (2000), the entire teachings of each of the foregoing are incorporated herein by reference). After the PCR reaction, the samples were stored at 4° C. The PCR products were examined using standard agarose gel electrophoresis to confirm that the primers yielded PCR products of the predicted size. Quantitative analysis was performed by scanning the gels (Hewlett-Packard ScanJet, Palo Alto, Calif.) and analyzing by segment area using UNSCAN-IT software (Silk Scientific, Orem Utah). The results of fkn and CX3CR1 expression were normalized to the expression of glyceraldehyde-3-phosphate dehydrogenase (GAPDH).

Statistical Analysis

The Students t-test was used to compare groups. Values of p≦0.05 were considered significant.

Results

Kinetics of Rat AIA Ankle Circumference and Cellular fkn and CX3CR1 Expression

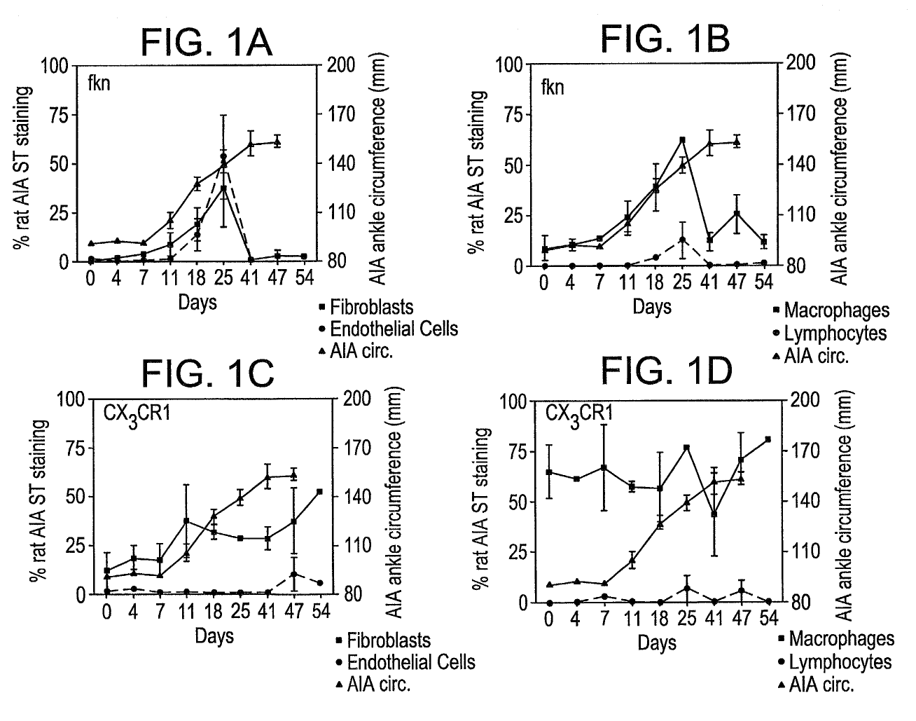

Rats were administered Complete Freunds Adjuvant (CFA) at the base of the tail and subsequently developed systemic arthritis. Most of the rats (90%) developed AIA by day 14 post-adjuvant injection. A continuous joint swelling was observed in the hind limbs beginning after day 7, which plateaued by day 41 (FIGS. 1A-1D, n=3 rats for each time point).

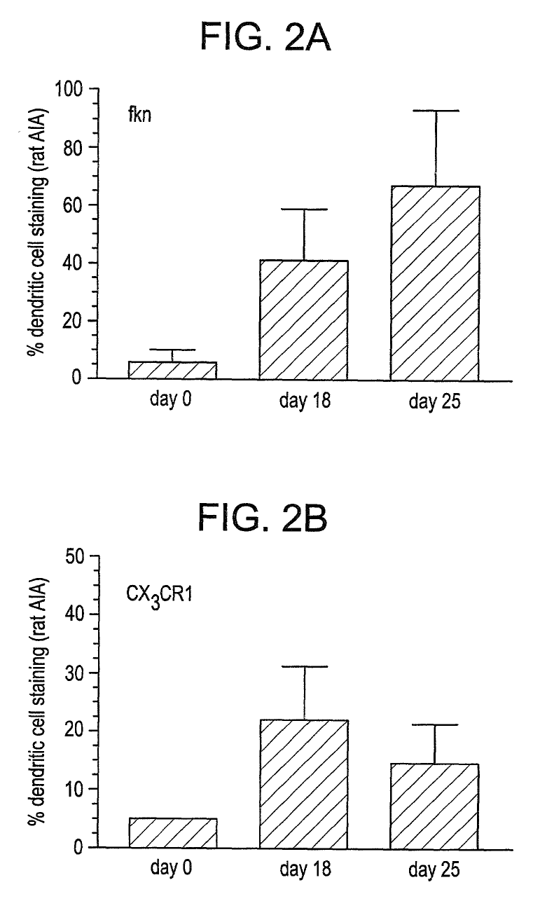

The kinetics of cellular fkn and CX3CR1 expression were identified by immunohistochemistry. FIGS. 1A and 1B shows the percentage of fkn immunopositive cells in rat AIA ST. A high percentage of macrophages (mean of 65%) (FIG. 1D) and fibroblasts (mean of 30%) (FIG. 1C) showed constitutive expression of CX3CR1, but a much lower percentage of lymphocytes (mean of 2%) (FIG. 1D) and endothelial cells (mean of 2%) expressed fkn (FIG. 1C). A greater percentage of fibroblasts (FIG. 1A) and macrophages (FIG. 1B) showed noticeably higher fkn expression on days 18 and 25, a time of maximal inflammation in the rat joint ((day 18 fibroblasts, 19%+8.6, mean+S.E.); (day 25 fibroblasts, 37.3%+19.7, mean+S.E.)), ((day 18 macrophages, 38.3%+11.7, mean+S.E.), (day 25 macrophages, 61.7%+1.7 mean+S.E.)). The highest percentage of endothelial cells also expressed fkn on days 18 and 25 (panel A) ((day 18 endothelial cells, 13.3%+8.3, mean+S.E.), (day 25 endothelial cells, 53.3%+21.3, mean+S.E.)) whereas lymphocytes did not significantly immunostain for fkn or CX3CR1 in rat AIA at any of the time points examined (panels B and D). FIGS. 2A and 2B show the percentage of dendritic cells that expressed fkn and CX3CR1 in rat AIA at days 18 and 25, compared to the percentage of dendritic cells that expressed fkn and CX3CR1 normal rats (day 0). The percentage of dendritic cell staining for both fkn and CX3CR1 is elevated at days 18 and 25 compared to normal rats ((day 0 dendritic cells for fkn, 5.5%+4.5, mean+S.E., n=2), (day 18 dendritic cells for fkn, 41.3%+17.4, mean+S.E., n=4), (day 25 dendritic cells for fkn, 66.7%+26.2, mean+S.E., n=3)), ((day 0 dendritic cells for CX3CR1, 5%+0, mean+S.E., n=2), (day 18 dendritic cells for CX3CR1, 21%+9,3, mean+S.E., n=3), (day 25 dendritic cells for CX3CR1, 14.3%+6.6, mean+S.E., n=4)).

Fkn and CX3CR1 Expression in Human RA ST

Immunohistochemical analysis of RA ST for fractalkine expression revealed that scattered throughout the synovium were a large number of intensely immunoreactive fusiform to stellate cells that extended long, slender cytoplasmic processes into the adjacent interstitum and were identified as dendritic cells, often surrounding individual or groups of positively-stained dendritic cells. The endothelium and synovial lining layers were intensely and diffusely immunoreactive for fractalkine, respectively. Immunohistochemical analysis of RA ST for CX3CR1 IgG expression revealed that scattered throughout the synovial connective tissue were abundant large, polyhedral to fusiform cells with abundant cytoplasm that were immunoreactive for CX3CR1. These cells appeared to be macrophages based on morphology, and were CD68⁺ in serial sections. CX3CR1-positive dendritic cells were also identified, with the majority of cells within the synovial lining layer being immunoreactive for CX3CR1. However, moderate numbers of low cuboidal to polyhedral cells with abundant, foamy pale basophilic cytoplasm, which were discreetly unstained, were also scattered throughout the synovial lining layer.

Expression of fkn and CX3CR1 on Human PB and SF CD14+ Monocytes and CD3+ T Cells by Flow Cytometry

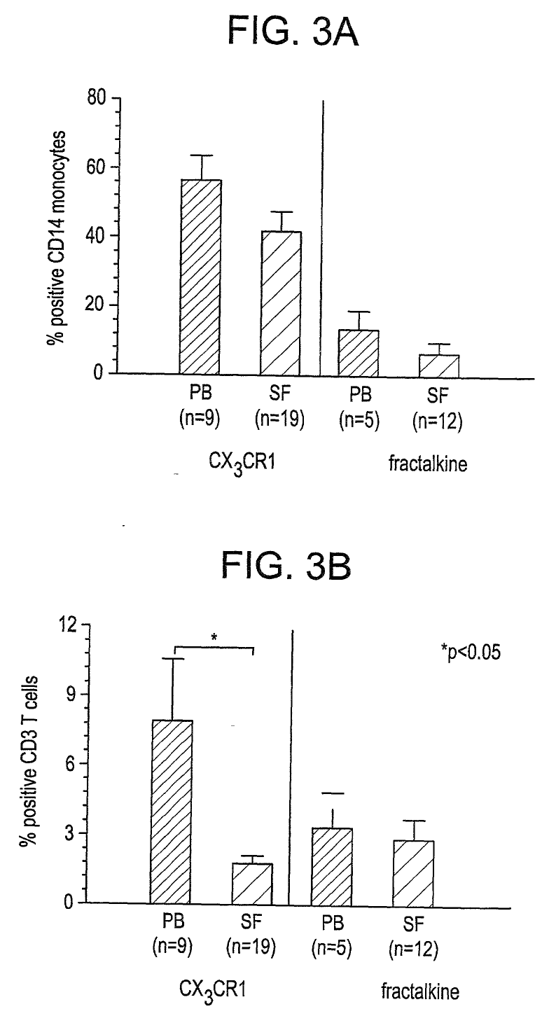

A modest percentage of PB and SF T-cells (PB 3%, n=5; SF 3%, n=12) and monocytes (PB 14%, n=5; SF 7%, n=12) expressed membrane associated fkn by flow cytometry (FIGS. 3A and 3B). The percentage of PB T cells expressing CX3CR1 (8%, n=9) was significantly increased compared to RA SF T-lymphocytes (2%, n=19). Both RA PB and SF monocytes expressed CX3CR1 (PB 56%, n=9; SF 42%, n=19). Flow cytometric analysis was done on multiple samples collected from different RA patients (5 PB and 13 SFS).

Data were also collected on a number of clinical parameters from patients with rheumatoid arthritis (RA). Table 1 shows the results of some statistically-significant correlations relating several variables to fkn and CX3CR1 expression on CD14+ and CD3+ cells. Specifically, RA PB CD3+ cells correlated positively with CX3CR1 expression and the age of the patient (r=0.51, n=16, p<0.05), and RA SF CD3+ cells correlated positively with CX3CR1 and patient swollen joint count (SJC) (r=0.75, n=l 5, p<0.05). With respect to monocytes, RA SF CD14+ cells correlated positively with fkn expression and early morning stiffness (EMS) (r=1.0, n=5, p<0.05). RA SF CD14+ CX3CR1 expression inversely correlated with erythrocyte sedimentation rate (ESR) (r=−0.013, n=12, p<0.05).

TABLE 1
Patient data correlating fkn and CX3CR1 expression

| | | | | |
|---|---|---|---|---|
| sample | RA PB | RA SF | RA SF | RA SF |
| cell type | CD3+ | CD3+ | CD14+ | CD14+ |
| antigen | CX3CR1 | CX3CR1 | fkn | CX3CR1 |
| variable | Age | SJC | EMS | ESR |
| r-value | r = 0.51 | r = 0.75 | r = 1.0 | r = 0.013 |
| nos. of obs. | n = 16 | n = 15 | n = 5 | n = 12 |
| p-value | p < 0.05 | p < 0.05 | p < 0.05 | p < 0.05 |

SJC = swollen joint count, EMS = early morning stiffness, ESR = erythrocyte sedimentation rate, nos. of obs. = number of observations.

Soluble fkn is Increased in RA SFs as Measured by ELISA

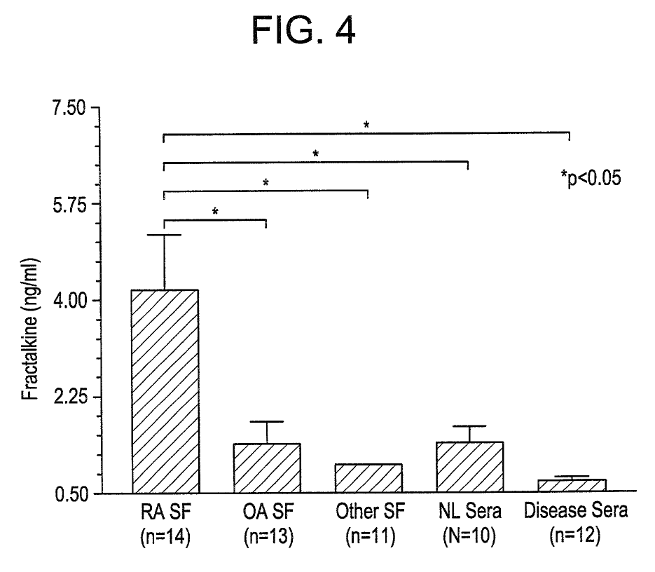

sfkn was measured by ELISA (FIG. 4). RA SFs had significantly elevated levels of sfkn (4.2+1.0 ng/ml, mean+S.E., n=14) compared to sera taken from healthy donors (1.4+0.3 ng/ml, mean+S.E., n=10), and sera from patients diagnosed with arthritic diseases ((0.71+0.07 ng/ml, mean+S.E., n=12), RA (n=5), OA (n=1), JRA (n=1), PSA (n=1), PA (n=2), and gout (n=2)). None of the RA serum samples contained detectable sfkn (≧500 pg/ml). SFs from OA patients (1.4+0.4 ng/ml, mean+S.E., n=13), or from individuals diagnosed with other diseases ((1.0+0.0 ng/ml, mean+S.E., n−11), JRA (n=2), PSA (n=3), PA (n=1), ANK (n=2), IM (n=1), and gout (n=2)), contained significantly lower sfkn levels than found in RA SF (p<0.05).

sfkn Contributes Significantly to RA SF Induced Mononuclear Cell Chemotaxis

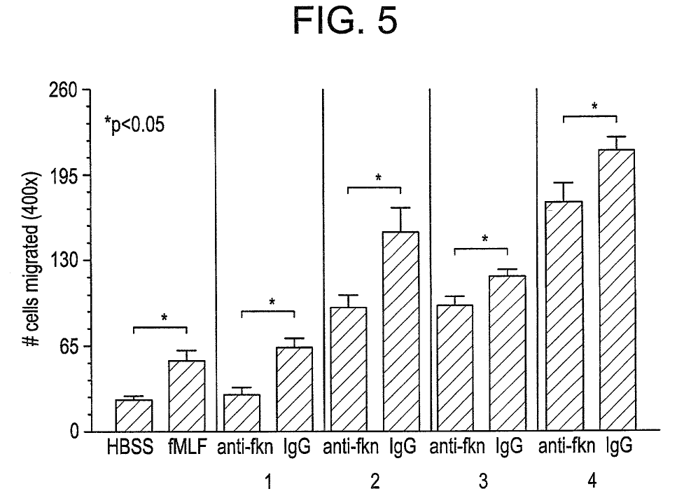

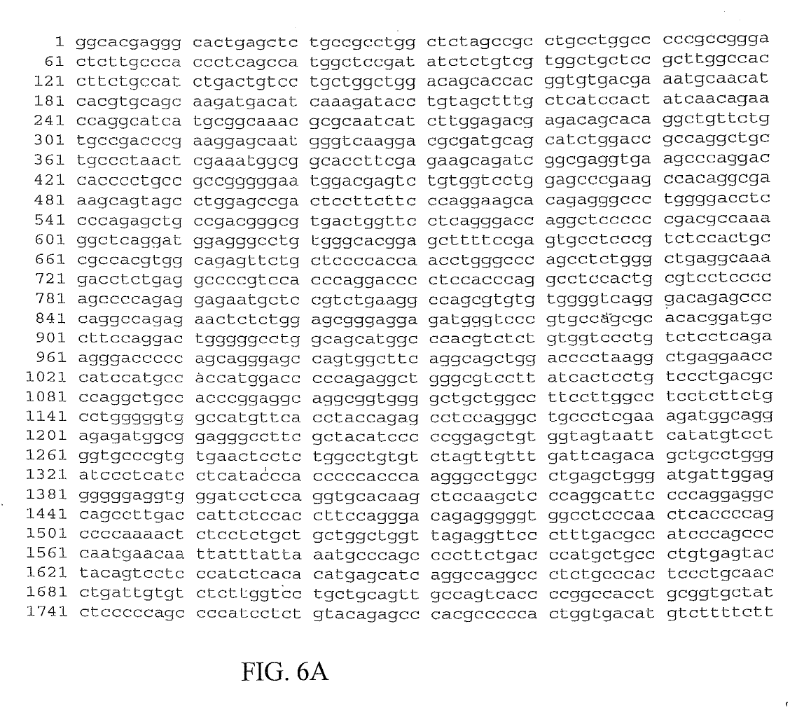

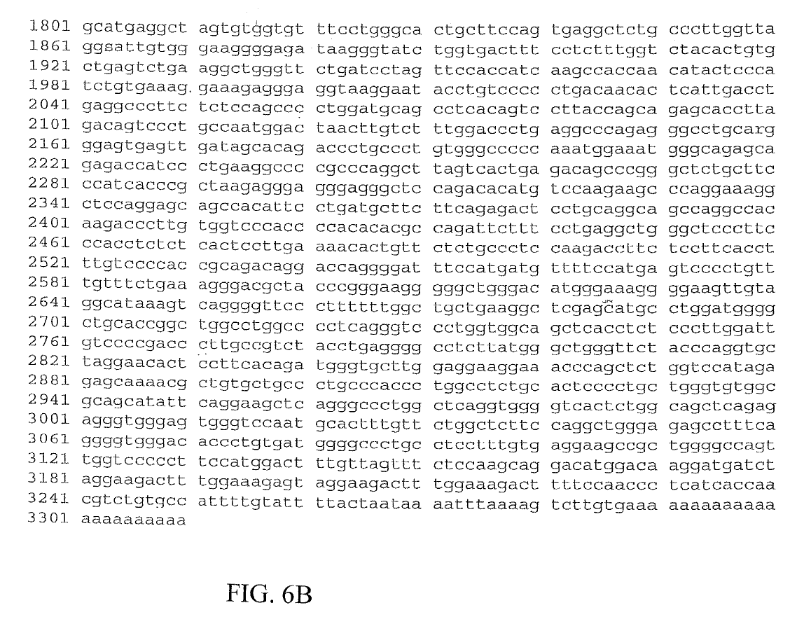

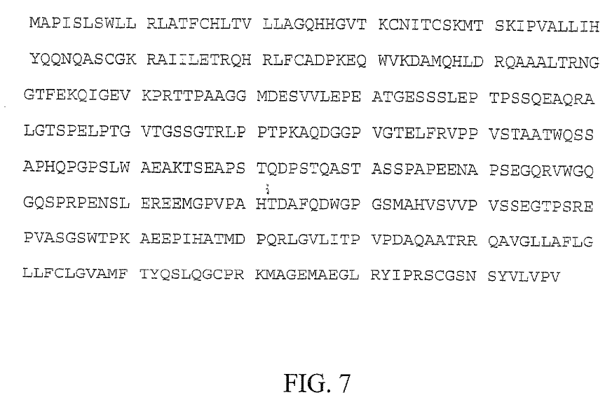

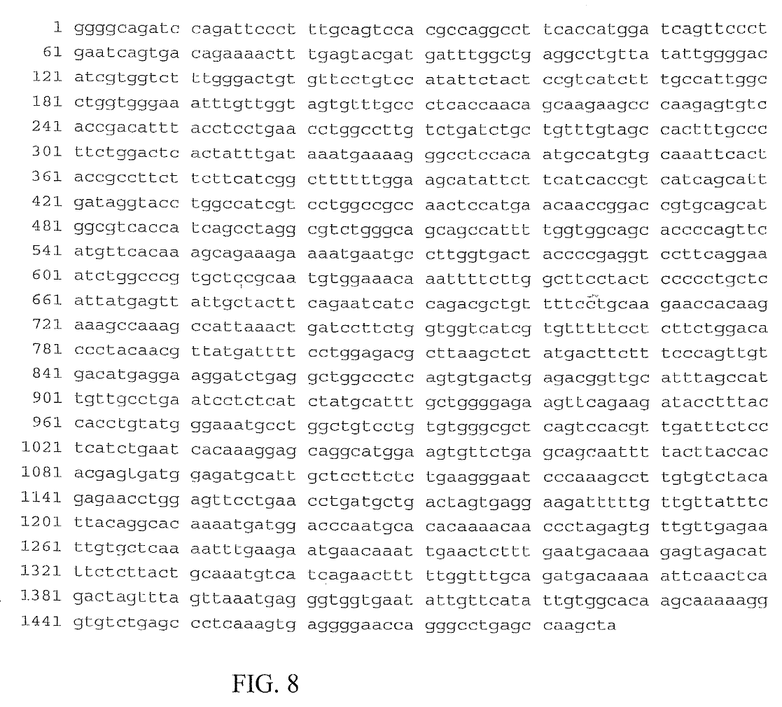

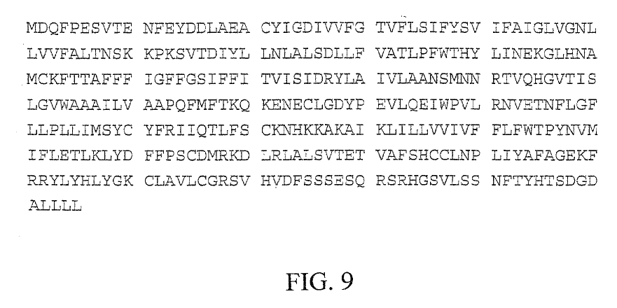

The contribution of sfkn to mononuclear chemotaxis (and infiltration of joints in RA) was determined by immunodepletion of RA SFs with polyclonal goat anti-human fkn IgG (anti-fkn) antibody, and used as “stimulant” in chemotaxis assays. The monocytes that migrated in a 400×field (done in quadruplicate) were counted. SFs depleted of sfkn from four different RA patients (FIG. 5) show impaired ability to chemoattract monocytes (overall 32% inhibition) compared to sham-depleted RA SFs (ns IgG), (RA patient 1 (anti-fkn 30+4.4 cells; ns IgG 65+6.6 cells, p,0.05), RA patient 2 (anti-fkn IgG 95+9.0 cells; ns IgG 151+18.6 cells, p<0.05), RA patient 3 (anti-fkn IgG 96+6.6 cells; ns IgG 116+5.3 cells, p<0.05), RA patient 4 (anti-fkn IgG 173+15.1 cells; ns IgG 213+10.6 cells, p<0.05)). Hanks Balanced Salt solution (HBSS, 25+3.2 cells) and fMLF ( 56+7.2 cells) were negative and positive controls respectively. Results were expressed as mean cells migrated+S.E.

Expression of fkn and CX3CR1 mRNA in Inflamed Rat Tissue

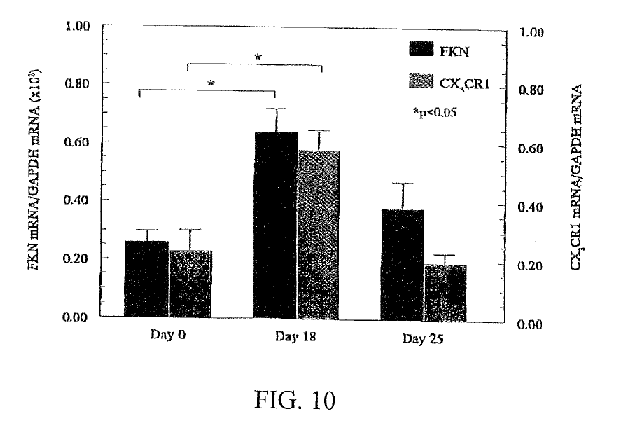

Total RNA was isolated from inflamed rat ankle homogenates and reverse transcribed into cDNA. cDNA was amplified using specific synthetic oligonucleotide primers for rat fkn (day 0, n=5; day 18, n=5; day 25, n=7 rat joints) and rat CX3CR1 (day 0, n=5; day 18, n=4; day 25, n=7 rat joints) and then quantified and normalized to the results obtained using synthetic oligonucleotide primers for glyceraldehyde-3-phosphate dehydrogenase (GAPDH). As shown in FIG. 10, the expression of fractalkine (fkn) and CX3CR1 in ankle homogenates from rats in which adjuvant-induced arthritis (AIA) had been induced, was significantly upregulated 18 days after administration of adjuvant (*p<0.05, significantly different from expression at day 0).

Discussion

This study defined the expression of fkn and CX3CR1 in the RA joint. Immunohistochemical analysis was employed to define the expression of fkn and CX3CR1 in a rat AIA kinetic study. In rat AIA, no significant or discernable staining pattern for fkn and CX3CR1 on smooth muscle cells or polymorphonuclear cells was observed. However, a high percentage of ST macrophages and fibroblast cells stained positively for fkn and CX3CR1, noticeably on days 18 and 25 following administration of adjuvant, a period of maximal inflammation and cellular recruitment in the rat joint. The percentage of rat AIA ST fibroblast and macrophage cells expressing CX3CR1 increased throughout the entire study period (though day 54), although the rat ankle swelling began to plateau by day 41.

A large percentage of endothelial cells expressed high levels of fkn, but minimal CX3CR1, on days 18 and 25. This observation is consistent with Feng et al. who recently showed up-regulated endothelial fkn expression on nephritic rat glomeruli in a Wistar-Kyoto (WKY) crescentic glomerulonephritis model (Feng, L. et al., Kidney. Int., 56:612 (1999)). Consistent with the observations in rat AIA, the endothelium of human RA ST stained positively for fkn. RA ST macrophages were intensely positive for CX3CR1 and endothelial cells did not stain for CX3CR1. In rat AIA, extensive fkn staining was observed on RA ST dendritic cells, with increased percentages of dendritic cells expressing fkn and CX3CR1 on days 18 and 25. The finding of dendritic cell fkn expression concurs with previous reports which have described increased fkn expression on maturing dendritic cells where fkn was shown to be a potent adhesion molecule, and also thought to play a part in antigen presenting cell and T-cell communication (Papadopoulos, E. et al., Eur. J. Immunol., 29:2551 (1999)). Kanazawa et al. (Kanazawa, N. et al., Eur. J. Immunol., 29:1925 (1999)) also showed fkn expression by dendritic cells, and provided evidence that they may be an important source of fkn that could be largely responsible for T-cell recruitment.

Flow cytometric studies revealed comparable percentages of monocytes expressing fkn in RA PB and SF. A similar trend was found when the percentage of RA PB and SF monocytes expressing CX3CR1 were compared. No difference in the percentage of PB or SF T-cells expressing fkn was found. However, a significantly higher percentage of RA PB T-cells expressed CX3CR1 compared to SF T-cells, with the overall percentage of monocytes expressing either fkn or CX3CR1 in RA PB and SF consistently surpassing T-cells. Also, the percentage of monocytes that expressed CX3CR1 was greater than the percentage that expressed fkn. These observations illustrate the importance of monocytes in fkn-mediated inflammation, and are in agreement with the data obtained in the rat AIA immunohistochemical studies where a consistently higher percentage of macrophages expressed CX3CR1, than fkn, at all time points measured. It is possible that only low levels of sfkn actually find their way into the bloodstream, therefore requiring a large percentage of macrophages to express CX3CR1 in order to mount an effective immune response. Activated PB T-lymphocytes may behave in a similar fashion. The ELISA data supports this since sfkn was not detected in any of the five RA PB samples measured (assay sensitivity≧500 pg/ml), but was significantly higher in SF from patients with RA. We therefore visualize a scenario where sfkn is released during inflammation, and is in turn taken up by activated blood monocytes and T-lymphocytes. This would aid activated inflammatory cells to home to the inflammatory site. It is equally plausible however that CX3CR1 may have many ligands, which suggests a very complex in vivo situation where the inflammatory outcome could be influenced by the regulation of cellular CX3CR1 expression (and other receptors) and many promiscuous ligands. It is noteworthy that there was downregulation in the percentage of RA SF T-cells expressing CX3CR1 compared to T-cells located in the PB. As the T-cell infiltrates the synovium, it likely encounters other cytokines and chemokines which are locally produced. This interaction of T-cells with other cytokines may effectively downregulate infiltrating T-cell CX3CR1 expression. Conversely, we do not find any clear-cut differences in monocyte CX3CR1 expression from either RA PB or SF. This may reflect differences in T-cell and monocyte activation during arthritis, and would support the notion that these cell type fulfill different roles in vivo during inflammation (Chensue, S. et al., J. Immunol., 154:5969 (1995)).

Soluble fkn was measured in normal sera, and in sera and SFs taken from a diverse arthritic patient population. Increased amounts of sfkn were found in the SFs from RA patients compared to all other SFs measured. Soluble fractalkine significantly contributes to the RA SF chemotactic activity for monocytes as immunodepleting RA SF sfkn inhibited monocyte chemotaxis. The data from chemotaxis assays using four separate patient samples revealed an overall mean of 32% inhibition of SF-derived monocyte chemotactic activity after depletion of soluble fractalkine. The chemotaxis data illustrate the potent chemoattractant activity of sfkn for inflammatory cells, and strongly suggest that sfkn is an important contributor to monocyte chemotaxis in the RA joint.

Patient data demonstrated that fkn and CX3CR1 are associated with the pathology of RA. RA SF CD14+ fkn expression positively correlated with early morning stiffness (EMS) in the joints of RA patients. Moreover, RA SF CD3+ CX3CR1 expression positively correlated with patient swollen joint count (SJC).

This study identifies fkn and its receptors (e.g., CX3CR1) as significant mediators of the immunopathogenesis of inflammatory arthritis (e.g., rheumatoid arthritis).

### EXAMPLE 2

Angiogenesis is an important aspect of the vasculoproliferation found in the rheumatoid arthritic (RA) pannus. To determine if fractalkine can induce angiogenesis, in vitro and in vivo studies were performed. Recombinant human fractalkine significantly induced chemotaxis of human dermal microvascular endothelial cells (HMVECs), a facet of the angiogenic response, in the pM range in a concentration-dependent fashion (p<0.05). While basic fibroblast growth factor, a well-recognized angiogenic factor, induced HMVEC mitogenesis in vitro, fractalkine did not. The ability of fractalkine to induce tube formation on a Matrigel™ matrix in 8 well chamber slides was investigated. Expression of HMVEC mRNA for the fkn receptor, CX3CR1, was also identified. Fractalkine induced significantly more endothelial tubes/well than did a negative control (p<0.05). Fractalkine also induced significantly more blood vessel growth than did a suitable control (2.3-fold) into Matrigel™ plugs in vivo (p<0.05). These results clearly demonstrate that fractalkine can induce angiogenesis.

RA synovial fluid can induce angiogenesis. To determine if soluble fractalkine contributes to this activity, SFs from 6 RA patients were immunodepleted of soluble fractalkine. The depleted SFs induced 56% less chemotaxis of HMVECs than did sham-depleted RA SFs (p<0.05). In vivo, immunodepletion of fkn from 6 RA SFs significantly inhibited angiogenic activity in Matrigel™ plug assays (p<0.05).

The results of the study establish fractalkine as an angiogenic mediator and implicate fractalkine and its receptors (e.g., CX3CR1) in the pathogenic vasculoproliferation found a variety of conditions (e.g., rheumatoid arthritis, cancer).

Materials and Methods

Reagents

Human recombinant fractalkine (fkn), interleukin-8 (IL-8), epithelial neutrophil-activating protein-78 (ENA-78), basic fibroblast growth factor (bFGF), bovine acidic fibroblast growth factor (aFGF), goat polyclonal anti-human fractalkine antibody (pAb) and goat IgG control antibody were obtained from R & D Systems (Minneapolis, Minn.). Dimethyl sulfoxide (DMSO) and phorbol 12-myristate acetate (PMA) were obtained from Sigma (St. Louis, Mo.). Matrigel™ was purchased from Becton Dickinson (Bedford, Mass.).

Cells

Human dermal microvascular endothelial cells (HMVECs) were obtained from BioWhittaker (San Diego, Calif.) and were grown in endothelial cell growth medium (EGM, BioWhittaker) with 10% fetal bovine serum (FBS). Cell assays were performed in endothelial basal medium (EBM, BioWhittaker) supplemented with FBS and 0.1% gentamicin. THP-1 cells were obtained from the American Type Culture Collection (Manassas, Va., Accession No. TIB-202) and were grown in RPMI with 10% FBS.

Endothelial Cell Chemotaxis

HMVECs were cultured in EGM containing 10% FBS. Chemotaxis was performed in 48-well chemotaxis chambers using gelatin-coated polycarbonate membranes with an 8 μm pore size (Neuroprobe, Cabin John, Md.) as previously described (Koch et al., Nature 376:517-519 (1995) and Halloran et al., Pathobiol. 65:287-292 (1997)). HMVECs (2.5×10⁴ cells in 25 μl of EBM containing 0.1% FBS) were added to the bottom wells. The chambers were inverted and incubated for 2 hours at 37° C. allowing HMVEC attachment to the membrane. Fractalkine (10¹²-10²nM), phosphate-buffered saline (PBS) or positive-control basic fibroblast growth factor (bFGF, 60 nM) were added to the top wells and the chambers were incubated for 2 hours at 37° C. The membranes were removed, fixed in methanol and stained with Diff-Quik (Baxter Scientific, Deerfield, Ill.). The number of cells that had migrated through the pores in the filter was counted per three high power fields and each test group was assayed in quadruplicate.

Immunodepletion of Fractalkine in HMVEC Chemotaxis Assays

Fractalkine (10¹ nM and 10⁻³ mM) or PMA (60 nM) was incubated with 10-25 μg/ml of either goat polyclonal anti-human fractalkine antibody (pAb) or control goat IgG antibody for 1 hour at 37° C. Upon completion of this neutralized period, the fkn/antibody and PMA/antibody combinations were assayed using the HMVEC chemotaxis assay described herein.

Immunodepletion of Fractalkine in RA SFs for HMVEC Chemotaxis Assays

Synovial fluids (SFs) were isolated from 6 patients with rheumatoid arthritis (RA) during therapeutic arthrocentesis. RA SF was diluted 1 to 50 with phosphate-buffered saline (PBS) and pre-incubated with 25 μg/ml of goat polyclonal anti-human fractalkine antibody (pAb) or goat IgG control antibody for 1 hour at 37° C. Upon completion of this neutralization period, the RA SF/antibody combinations were assayed using the HMVEC chemotaxis assay described herein.

Formation of Endothelial Cell (EC) Tubes on Matrigel™ in vitro

Matrigel™ was thawed on ice to prevent premature polymerization; 125 μl was plated into individual wells of eight-well chamber slides (Falcon, Bedford, Mass.) and allowed to polymerize at 37° C. for 30-60 minutes. HMVECs were removed from culture by trypsinization and resuspended at 4×10⁴ cells/ml in Medium 199 (Gibco BRL, Grand Island, N.Y.) containing 2% FBS and 200 μg/ml of EC growth supplement, as previously described (Schnaper et al., J. Cell Physiol. 156:235-246 (1993)). 400 μl of cell suspension containing fractalkine, 50 nM PMA or vehicle control (PBS for fkn, PBS and DMSO for PMA) was plated in each well and plates were incubated for 16-18 hours at 37° C. in a 5% CO₂-humidified atmosphere (Kinsella et al., Exp. Cell Res. 199:56-62 (1992)). Culture medium was aspirated off and cells were fixed with Diff-Quick Fixative and stained with Diff-Quick Solution II (Baxter Scientific, Deerfield, Ill.). Each chamber was photographed using a Polaroid Microcam camera at a final magnification of 22×. The number of tube branches was quantified by a blinded observer (Gately et al., Cancer Res. 56:4887-4890 (1996)). Each concentration of control or test substance was assayed in triplicate.

Human Dermal Microvascular Endothelial Cell (HMVEC) Proliferation Assay

HMVEC proliferation was quantified using a CellTiter 96® Aqᵤₑₒᵤₛ Assay (Promega, Madison, Wis.) (Koch et al., Nature 376:517-519 (1995) and Halloran et al., Pathobiol. 65:287-292 (1997)). HMVECs in EBM+2% FBS+0.1% gentamicin were plated in 96-well plates (2,500 cells/well) for 4 hours, allowing cells to adhere to the plates. The test substances, diluted in medium, were added to the appropriate wells and incubated at the manufactures' suggested conditions of 37° C. and 5% CO₂ for 72 hours. Following the incubation, viable cells were detected by reading their absorbance at a wavelength of 490 nm. These values were compared to a positive control, basic fibroblast growth factor (bFGF), and a negative control, medium alone.

Reverse Transcriptase-PCR (RT-PCR) Amplification of HMVEC CX3CR1

HMVECs were cultured in endothelial cell growth medium (EGM, BioWhittaker) containing 10% FBS. Total RNA (1 μg) was prepared from HMVECs and first strand cDNAs were synthesized using an oligo dT primer and AMV reverse transcriptase (Promega, Madison, Wis.). Subsequent amplification of CX3CR1 from HMVEC cDNA was performed using synthetic oligonucleotide 5′ and 3′ primers: forward primer 5′ ctctatgacttctttcccagttgt 3′ (SEQ ID NO:11); reverse primer 5′ agacacaaggctttgggattc 3′ (SEQ ID NO:12). PCR cycling conditions were 95° C. for 5 minutes followed by 30 cycles of 95° C. for 1 minute, 52° C. for 1 minute and 72° C. for 1 minute and ended by 10 minutes at 72° C. Amplification products were characterized using size fractionation on 1% agarose gels.

Matrigel™ Plug Assays for Angiogenesis in vivo

Female 8-12 week old C57BL/6 mice (Charles River Lab, Wilmington, Mass.) were each injected subcutaneously near their abdominal midline using a 30 gauge needle with 0.5 ml of Matrigel™ combined with either PBS, fractalkine (100 nM), interleukin-8 (IL-8, 100 nM), epithelial neutrophil-activating protein-78 (ENA-78, 100 nM) or positive control bovine acidic fibroblast growth factor (aFGF, 63 pM) (Passaniti et al., Lab Invest. 67:519-528 (1992) and Johns et al., Endocrinology 137:4511-4513 (1996)). Seven to ten days later, the mice were sacrificed and the Matrigel™ plugs were removed, weighed and processed for histology or hemoglobin concentration determination. For histological analysis, plugs were formalin-fixed, paraffin-embedded, cut into 4 μm sections and Masson trichrome stained. For hemoglobin determination, which correlates with the number of blood vessels, plugs were homogenized in 1 ml of distilled water. Hemoglobin concentration was determined either by the Drabkin method using a Drabkin's Reagent Kit (Sigma) or using a 3,3′,5,5′-tetramethylbenzidine liquid substrate system (Sigma).

Immunodepletion of Fractalkine in RA SFs for Matrigel™ Plug Angiogenesis Assays

Synovila fluids (SFs) were isolated from 6 patients with rheumatoid arthritis (RA) during therapeutic arthrocentesis. RA SFs were pooled and diluted 1 to 10 PBS and pre-incubated with 25 μg/ml of goat polyclonal anti-human fractalkine antibody (pAb) or goat IgG control antibody for 1 hour at 37° C. Upon completion of this neutralized period, the RA SF/antibody combination was diluted again 1 to 10 with Matrigel™ and assayed using the in vivo Matrigel™ plug angiogenesis assay described herein.

Immunodepletion of Fractalkine in RA STs for Matrigel™ Plug Angiogenesis Assays

Synovial tissues (STs) were obtained from 5 patients undergoing total joint replacement who met the American College of Rheumatology criteria for rheumatoid arthritis (RA) (Altman et al., Arthritis Rheum. 29:1039-1049 (1986), Altman et al., Arthritis Rheum. 34:505-514 (1991) and Arnett et al., Arthritis Rheum. 31:315-324 (1988)). RA STs were homogenized in 1 ml of an “anti-protease” buffer as described (Keane et al., J. Immunol. 159:1437-1443 (1997)). Samples were sonicated, centrifuged at 900 g for 15 minutes and filtered through a 1.2-μm pore size sterile Acrodisk (Gelman Sciences, Ann Arbor, Mich.) and frozen at −80° C. until thawed for assay. ST homogenates were thawed, normalized, pooled and pre-incubated with 25 μg/ml of goat polyclonal anti-human fractalkine antibody (pAb) or goat IgG control antibody for 1 hour at 37° C. Upon completion of this neutralization period, the RA ST homogenates/antibody combinations were diluted 1 to 25 with Matrigel™ and assayed using the in vivo Matrigel™ plug angiogenesis assay described herein.

Statistical Analysis

Data were analyzed using Student's t-test. p values less than 0.05 were considered significant.

Results

Fractalkine Induces HMVEC Chemotaxis in vitro

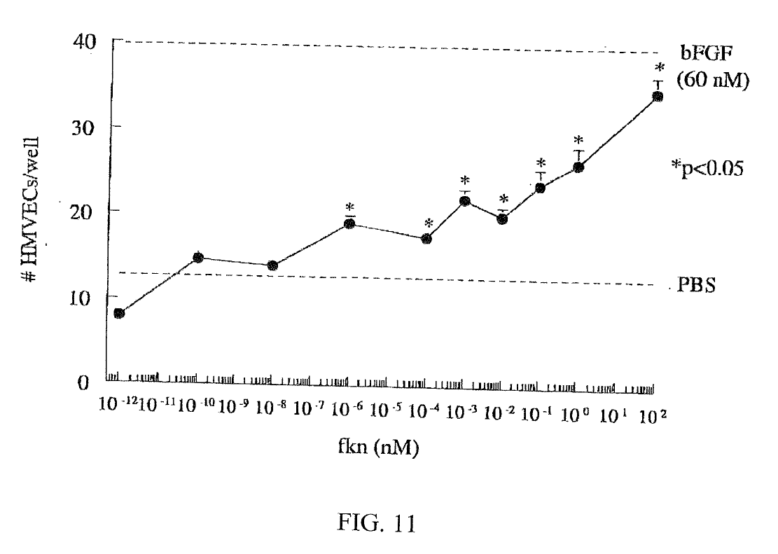

Fractalkine was assayed for its ability to induce HMVEC chemotaxis in vitro. Results of a representative experiment of four are shown in FIG. 11. Fkn induced chemotaxis in a concentration-dependent fashion in the pM and nM concentration range. Fkn (10⁻³ pM-10² nM) significantly increased endothelial cell (EC) chemotaxis over negative control PBS (p<0.05).

Fractalkine-induced Chemotactic Activity for HMVECs is Decreased by Immunodepletion of Fractalkine

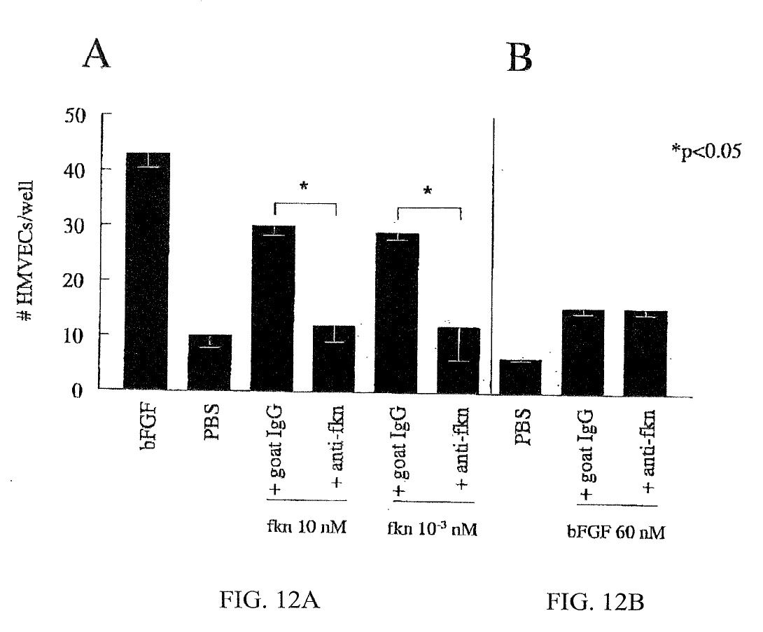

Fractalkine was incubated with 25 μg/ml of an antibody specific for the CX3C chemokine domain of fkn (goat polyclonal anti-human fractalkine antibody, pAb) and then assayed for HMVEC chemotaxis ability. FIG. 12A shows that at concentrations from 1 pM to 10 nM of fkn, the anti-CX3C domain antibody completely inhibited fkn-induced HMVEC chemotaxis (p<0.05). This inhibition of chemotaxis by an anti-CX3C domain antibody was specific for fkn-induced HMVEC chemotaxis as basic fibroblast growth factor (bFGF)-induced chemotaxis was not affected by incubation with this antibody (FIG. 12B).

Fractalkine Does Not Induce HMVEC Proliferation in vitro

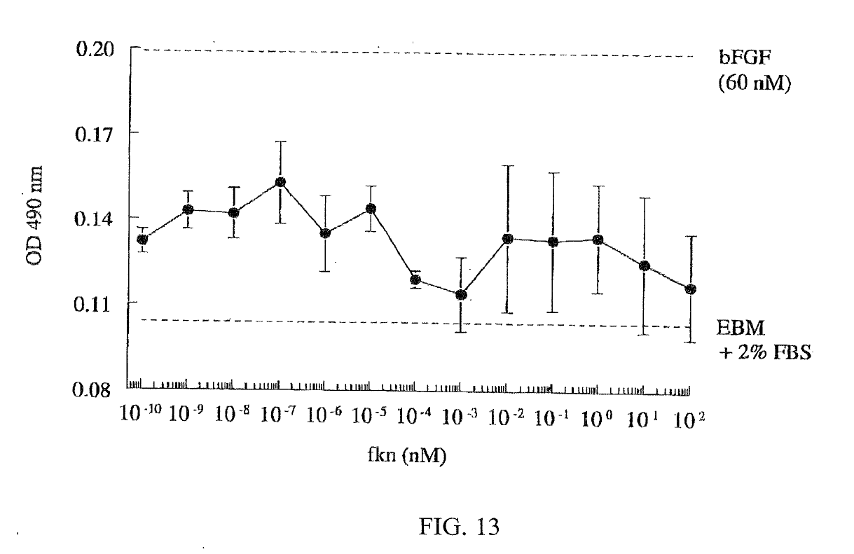

The ability of fractalkine to act as a mitogen for HMVECs in vitro was also examined. When assayed in concentrations of 10⁻¹⁰ to 10² nM, fkn did not induce a mitogenic response, in contrast to basic fibroblast growth factor (bFGF, 60 nM) which induced potent EC proliferation. Results of a representative experiment of four experiments are shown in FIG. 13.

Fractalkine-induced HMVEC Tube Formation on Matrigel™ in vitro

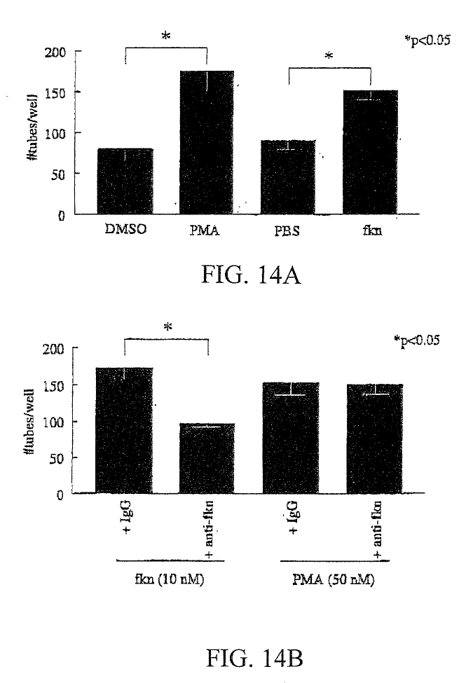

Tube formation, one facet of the angiogenic response, can be assayed in vitro by testing the ability of HMVECs, which are plated on Matrigel™, to form tubes. The ability of fkn to induce tube formation on Matrigel™ was tested using eight well chamber slides. In order to quantify tube formation in the Matrigel™ matrices, a blinded observer counted endothelial cell (EC) tubes in each experimental well. FIG. 14A shows EC tube counts for fkn-induced tube formation along with tube counts induced by positive control PMA and vehicle controls DMSO and PBS. Fkn induced significantly more EC tube formation than negative control PBS (152±11.7 vs 90±10.7 tubes/well) (p<0.05, n=4). As positive controls, the angiogenic chemokines interleukin-8 (IL-8) and epithelial neutrophil-activating protein-78 (ENA-78) were assayed and both induced EC tube formation to a greater extent than PBS controls. Addition of goat polyclonal anti-human fractalkine antibody (pAb) with specificity for the CX3C domain of fkn significantly inhibited fkn-induced tube formation over isotype control antibody (p<0.05, n=4), thereby demonstrating that fkn-induced tube formation was due to the chemokine domain, and not the mucin stalk domain, of fkn (FIG. 14B). This inhibition of tube formation by anti-fkn antibody was specific to fkn-induced formation as PMA-induced tube formation was not affected by incubation with the anti-fkn antibody (FIG. 14B).

HMVECs Express mRNA for CX3CR1 in vitro

RT-PCR was performed on HMVEC cDNAs or on cDNAs from THP-1 cells, a myeloid cell line previously reported to express high amounts of CX3CR1 mRNA (Raport et al., Gene 163:295-299 (1995)). PCR products were synthesized using specific human CX3CR1 primers which amplify a 320 bp fragment. A 320 bp PCR product was amplified from both HMVEC cDNAs and positive control THP-1 cDNAs, indicating endothelial cell (EC) expression of CX3CR1.

Fractalkine-induced Angiogenesis in Matrigel™ Plugs in vivo

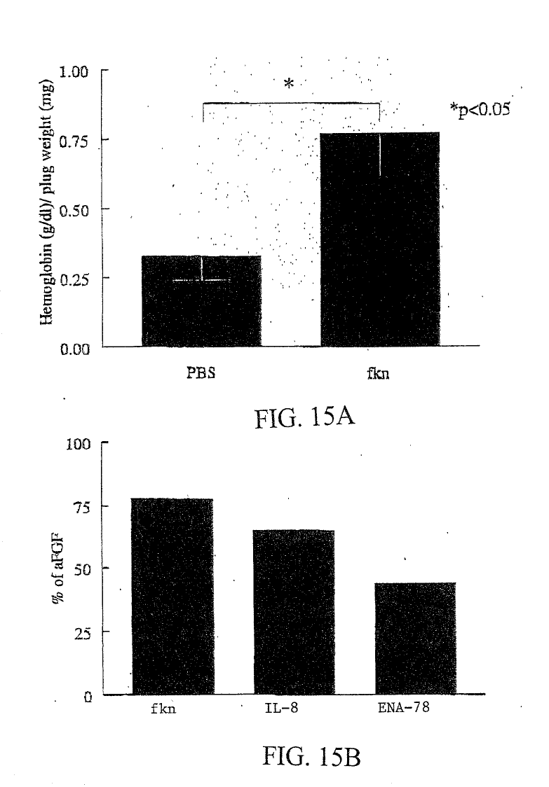

To determine whether fractalkine functions as an angiogenic mediator in vivo, we employed the mouse Matrigel™ plug assay. Matrigel™ plugs containing negative control PBS, fkn, angiogenic chemokines interleukin-8 (IL-8) or epithelial neutrophil-activating protein-78 (ENA-78), or positive control bovine acidic fibroblast growth factor (aFGF) were implanted subcutaneously into the abdomen of mice. Examination of fkn-containing plugs seven to ten days after implantation revealed marked new blood vessel growth while minimal blood vessel growth was induced by negative control PBS. FIG. 15A shows the hemoglobin content normalized to the weight of the Matrigel™ plugs for fractalkine- and PBS-containing Matrigel™ plugs. The hemoglobin content correlates with the number of blood vessels in the plugs. As shown in FIG. 15A, fkn induced significantly more blood vessels in the Matrigel™ plugs than did negative control PBS (0.77±0.15 versus 0.33±0.08 g/dl of hemoglobin/mg of plug weight, respectively, n=18, p<0.05). In order to compare the relative angiogenic potency of fkn to other known angiogenic chemokines, IL-8 and ENA-78 were also tested in the Matrigel™ plug model. The relative angiogenic potencies for fkn, IL-8, and ENA-78, as a percentage of the angiogenic potency of the positive control, aFGF, are shown in FIG. 15B. Fkn exhibited 78% of the angiogenic potency of aFGF, while IL-8 and ENA-78 exhibited 65% and 44% respectively (n=7-9).

RA SF Chemotactic Activity for HMVECs is Decreased by Immunodepletion of Fractalkine

Synovial fluids (SFs) from 6 patients with rheumatoid arthritis (RA) were immunodepleted of fractalkine and assayed for HMVEC chemotactic activity. Results of the immunodepletion studies are shown in Table 2. While RA SF was potently chemotactic for HMVECs, immunodepletion of fkn from RA SFs resulted in significantly decreased (56.1±2.4%, mean±S.E.) chemotactic activity for HMVECs relative to immunodepletion with an isotype control antibody (p<0.05).

TABLE 2
Migration of HMVECs in response to RA SF incubated in the presence
and absence of anti-fkn antibody.

| Patient | Goat IgG | Anti-fkn | % Suppression^{A} |
|---|---|---|---|
| 1 | 35 | 17 | 51.4 |
| 2 | 31 | 15 | 51.6 |
| 3 | 28 | 11 | 60.7 |
| 4 | 25 | 9 | 64.0 |
| 5 | 32 | 16 | 50.0 |
| 6 | 29 | 12 | 58.6 |
| RA SFs were assayed for their ability to induce migration of HMVECs. The results represent mean number of cells per well as measured in three high power fields (×400). Each sample was tested in 2 to 4 wells. The ability of goat polyclonal anti-human fractalkine antibody (anti-fkn, 25 μg/ml) to neutralize the migratory properties of RA SF was determined as a percent suppression of migration as compared to that of a goat IgG isotype control antibody (goat IgG) | | | |
| # (mean % Suppression = 56.1 ± 2.4). Positive control migration in response to basic fibroblast growth factor (bFGF, 60 nM) was a mean of 44 cells/well. Negative control migration in response to PBS was a mean of 12 cells/well. ^{A}p value for percent suppression compared to isotype IgG control antibody (goat IgG) in all patient samples assayed was <0.05. | | | |

RA SF Angiogenic Activity is Decreased by Immunodepletion of Fractalkine

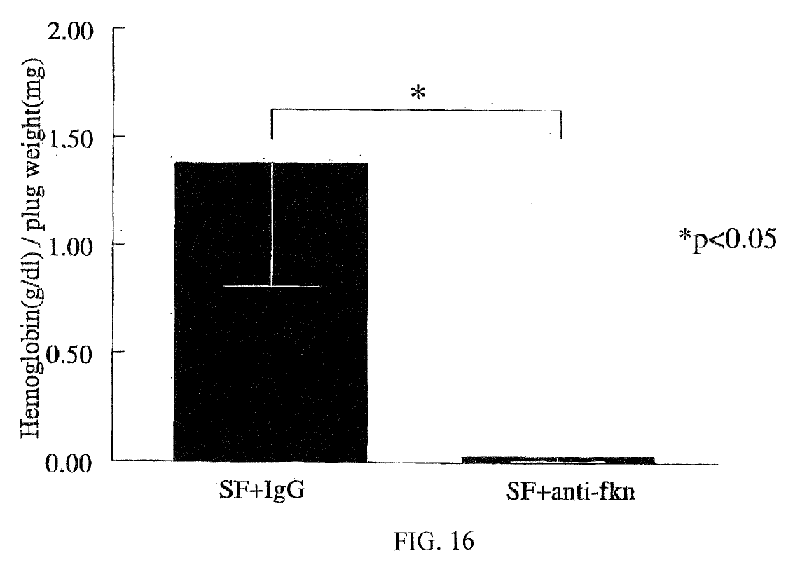

Synovial fluids (SFs) from 6 rheumatoid arthritis (RA) patients were pooled, immunodepleted of fractalkine and assayed for angiogenic activity in vivo. Fkn-immunodepleted SFs were diluted in Matrigel™ and injected subcutaneously into mice. Results of these immunodepletion experiments are shown in FIG. 16. Angiogenesis induced by the pooled SFs was significantly decreased in SFs immunodepleted of fkn as compared to SFs that were immunodepleted using an IgG control antibody (0.028+0.02 versus 1.38±0.57 g/dl of hemoglobin/mg of plug weight, respectively, n=12, p<0.05).

RA ST Angiogenic Activity is Decreased by Immunodepletion of fkn

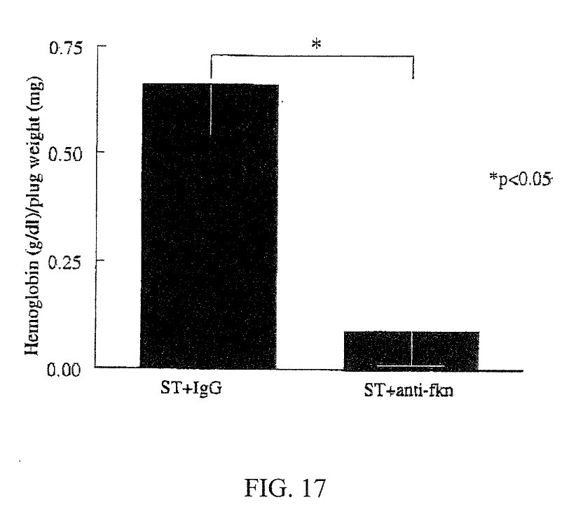

Synovial tissue (ST) homogenates from 5 rheumatoid arthritis (RA) patients were pooled, immunodepleted of fkn and assayed for in vivo angiogenic activity. Examination of Matrigel™ plugs containing pooled RA ST homogenates revealed significant new blood vessel growth, while Matrigel™ plugs containing RA ST homogenates that had been immunodepleted of fkn using goat polyclonal anti-human fractalkine antibody displayed minimal blood vessel growth. FIG. 17 shows the hemoglobin content normalized to the weight of the Matrigel™ plugs. The angiogenesis induced by the pooled ST homogenates was significantly decreased by immunodepleting fkn as compared to IgG-immunodepleted ST homogenates (0.09±0.08 versus 0.66±0.12 g/dl of hemoglobin/mg of plug weight, respectively, n=12, p<0.05).

Discussion

This study demonstrated that fractalkine functions as a chemoattractant for endothelial cells (ECs) and an inducer of angiogenesis. Fkn induced chemotaxis of HMVECs in a concentration-dependent manner from concentrations ranging from 10⁻⁶ nM to 10² nM, at which concentration it had similar activity to the potent EC chemoattractant basic fibroblast growth factor (bFGF, 60 nM) (FIG. 11).

In vitro assays also demonstrated that fkn (100 nM) induced endothelial cells to form tubes in Matrigel™ at the same rate as the angiogenic chemokines, interleukin-8 (IL-8) and epithelial neutrophil-activating protein-78 (ENA-78) (both at 10 μM), and PMA (50 μM), a strong inducer of endothelial cell differentiation and endothelial cell tube formation (FIG. 14A). The angiogenic properties of fkn were also demonstrated using in vivo assays. In these in vivo assays, fkn induced angiogenesis in Matrigel™ plugs inserted in mice to a comparable extent as the known angiogenic chemokines IL-8 and ENA-78 (FIG. 15B). Thus, fkn is the first CX3C chemokine shown to function as an inducer of endothelial cell chemotaxis and angiogenesis.

This study also demonstrated that the chemokine domain of fkn is necessary and that the mucin domain is not sufficient for inducing endothelial cell chemotaxis and tube formation on Matrigel™, as an antibody specific for the chemokine domain completely inhibited fkn-induced HMVEC chemotaxis and tube formation (FIGS. 12A and 14B). Studies also demonstrated that mRNA expression for the fkn receptor, CX3CR1, can be detected in HMVECs in culture.

The angiogenic properties of fkn are similar in potency to other angiogenic mediators. Fkn induced a doubling in the amount of endothelial cell chemotaxis, a technical indicator of potent chemotaxis, at 1 nM and reached statistical significance at concentrations as low as 10⁻⁶ nM. Fkn induced angiogenesis in vivo at 100 nM. These concentrations of fkn are comparable to concentrations of the CXC chemokines, IL-8, ENA-78, and growth-related oncogene α (GROα), which have been shown to induce endothelial cell chemotaxis and angiogenesis. For example, previous studies demonstrated that IL-8 induced a doubling in human umbilical vein EC chemotaxis at 1.25 nM and induced angiogenesis at 10 nM (Koch et al., Science 258:1798-1801 (1992)). Other studies have shown that ENA-78 induced bovine adrenal gland capillary endothelial cell chemotaxis at as low as 5 nM and ENA-78 and GROα induced angiogenesis in the rat cornea neovascularization assay at 10 nM (Strieter et al., J. Biol. Chem. 270:27348-27357 (1995)). Thus fractalkine is a powerful chemoattractant for endothelial cells and is angiogenic in vivo in the nM range, similar to other angiogenic CXC chemokines.

Angiogenic factors function to form intact microvessels by inducing endothelial cell migration, proliferation, elongation, orientation, and differentiation resulting in lumen formation, reestablishment of the basement membrane and anastomosis with other vessels. This study demonstrated that fkn induces endothelial cell chemotaxis, but not proliferation. In this way, fkn acts in the same fashion as other angiogenic mediators by inducing some facets of the angiogenic process. This study also demonstrated that fkn induced endothelial cells to form tubes on Matrigel™ in vitro and to form functional blood vessels in Matrigel™ plugs in vivo, thus establishing its angiogenic properties.

Synovial tissue from patients with rheumatoid arthritis (RA ST) is replete with newly formed blood vessels in response to the increased demand for nutrients and oxygen by the proliferating pannus tissue (Colville-Nash and Scott, Ann. Rheum. Dis. 51:919-925 (1992), Szekanecz et al., J. Invest. Med. 46:27-41 (1998) and Folkman, Nat Med. 1:27-31 (1995)). The level of RA ST vascularity correlates with more severe clinical and inflammatory scores and the degree of vascularity in RA ST is greater than that seen in osteoarthritis ST (Colville-Nash and Scott, Ann. Rheum. Dis. 51:919-925 (1992), Szekanecz et al., Arthritis Rheum. 37:221-231 (1994) and Johnson et al., Arthritis Rheum. 36:137-146 (1993)). As described herein (Example 1), RA SF and ST contain greater levels of angiogenic fkn than SF and ST from patients with osteoarthritis or other forms of arthritis. This study showed that RA SF which was immunodepleted of fkn had significantly reduced chemotactic activity for endothelial cells and that RA SF and ST homogenates which were immunodepleted of fkn had significantly reduced angiogenic activity.

Therefore, the results of this study show that fkn, the sole member of the CX3C chemokine family, induces endothelial cell chemotaxis, endothelial cell tube formation and blood vessel formation in vivo. Fractalkine is involved in the angiogenic activity of RA SF and ST homogenates. In a disease state such as rheumatoid arthritis, fkn can act in an autocrine fashion. Specifically, pro-inflammatory cytokines involved in rheumatoid arthritis (e.g., IL-1β, TNF-α) can activate endothelial cells to produce fkn on their surface. Endothelial cell surface fkn can be released (e.g., by enzymatic cleavage) and the resulting soluble fkn can bind endothelial cell CX3CR1 thereby inducing endothelial cell chemotaxis and synovial angiogenesis.

The results of the study establish fractalkine as an angiogenic mediator and implicate fractalkine and its receptors (e.g., CX3CR1) in the pathogenic vasculoproliferation found in a variety of conditions (e.g., rheumatoid arthritis, cancer).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting angiogenesis in a subject, comprising administering to said subject a therapeutically effective amount of an antagonist of CX3CR1 function.

2. A method of inhibiting angiogenesis in a subject, comprising administering to said subject a therapeutically effective amount of an antagonist of fractalkine function.

3. The method of claim 2 wherein said antagonist binds mammalian fractalkine and inhibits the binding of fractalkine to receptor.

4. The method of claim 3 wherein said receptor is CX3CR1.

5. A method of inhibiting angiogenesis in a subject, comprising administering to said subject a therapeutically effective amount of an agent which binds mammalian CX3CR1 and inhibits the binding of ligand to said CX3CR1.
